(19) United States
(12) Patent Application Publication
(10) Pub. No.: US 2025/0120692 A1
(43) Pub. Date: Apr. 17, 2025

(54) PROPORTIONATE BALANCING OF THE FUNCTION IMPACT MAGNITUDE OF BATTERY OUTPUT TO PEAK MOTOR CURRENT

(71) Applicant: Cilag GmbH International, Zug (CH)

(72) Inventors: Shane R. Adams, Lebanon, OH (US); Frederick E. Shelton, IV, Hillsboro, OH (US); Matthew D. Cowperthwait, Cincinnati, OH (US); Nicholas J. Ross, Franklin, OH (US); Kevin M. Fiebig, Cincinnati, OH (US); Eric B. LaFay, Madeira, OH (US); Taylor W. Aronhalt, Loveland, OH (US)

(21) Appl. No.: 18/379,768

(22) Filed: Oct. 13, 2023

(51) Int. Cl. A61B 17/068; A61B 17/00

(52) U.S. Cl. CPC A61B 17/068; A61B 2017/00199; A61B 2017/00398

(57) ABSTRACT

A surgical system is disclosed that includes a motor, a power source, a voltage sensor, and a control circuit. The motor is operable between an on state in which the motor drives a motion at the end effector and an off state in which the motor ceases to drive the motion at the end effector. The power source is electrically coupled to the motor. The control circuit is configured to transition the motor to the on state for a first period, detect a dropped voltage potential of the power source at the end of the first period, conduct a comparison between the dropped voltage potential and the power source lower threshold, and transition the motor to the off state for a second period based on the comparison.

### BACKGROUND

The present disclosure relates to surgical instruments and, in various arrangements, to surgical stapling and cutting instruments and staple cartridges for use therewith that are configured to staple and cut tissue.

### SUMMARY

The present disclosure provides a control system that implements micro and/or macro recoveries to a power source such that the power output of the power source can be maintained at a high level during use of a surgical cutting and stapling instrument.

In accordance with the present disclosure, a surgical system is disclosed that comprises an end effector, a motor, a power source, a voltage sensor, and a control circuit. The end effector includes a first jaw and a second jaw rotatable relative to the first jaw from an open position toward a closed position to capture tissue therebetween. The motor is operable between an on state in which the motor drives a motion at the end effector and an off state in which the motor ceases to drive the motion at the end effector. The power source is electrically coupled to the motor. The voltage sensor is configured to sense the voltage potential of the power source. The control circuit is in operable communication with the motor and the voltage sensor. The control circuit is configured to set a power source lower threshold, transition the motor to the on state for a first period, detect a dropped voltage potential of the power source at the end of the first period, conduct a first comparison between the dropped voltage potential and the power source lower threshold, and transition the motor to the off state for a second period based on the first comparison.

In accordance with the present disclosure, a surgical system is disclosed that comprises an end effector, a motor, a power source, a voltage sensor, and a control circuit. The end effector comprises a first jaw and a second jaw rotatable relative to the first jaw from an open position toward a closed position to capture tissue therebetween. The power source is configured to power the motor. The voltage sensor is configured to sense the voltage potential of the power source. The control circuit is in operable communication with the motor and the voltage sensor. The control circuit is configured to set a recovery threshold, receive a first input, control the motor to drive a first motion at the end effector based on receiving the first input, monitor a voltage potential of the power source based on the first motion concluding, receive a second input, compare the monitored voltage potential to the recovery threshold based on receiving the second input, and perform an action based on the comparison.

In accordance with the present disclosure, a surgical system is disclosed that comprises an end effector, a motor, a power source, a timer, and a control circuit. The end effector comprises a first jaw and a second jaw rotatable relative to the first jaw from an open position toward a closed position to capture tissue therebetween. The power source is configured to power the motor. The timer is configured to measure elapsed time. The control circuit is in operable communication with the motor and the voltage sensor. The control circuit is configured to receive a first input, control the motor to drive a first motion at the end effector based on receiving the first input, monitor an elapsed period based on the first motion concluding, receive a second input, compare the elapsed period to a recovery time period based on receiving the second input, and perform an action based on the comparison.

### BRIEF DESCRIPTION OF THE DRAWINGS

Various features of the embodiments described herein, together with advantages thereof, may be understood in accordance with the following description taken in conjunction with the accompanying drawings as follows:

FIG. 1 illustrates a perspective view of a surgical stapling system, in accordance with the present disclosure;

FIG. 2 illustrates an exploded view of the surgical stapling system of FIG. 1, in accordance with the present disclosure;

FIG. 3 illustrates a block diagram of a surgical stapling system, in accordance with the present disclosure;

FIG. 4 illustrates a block diagram of a surgical stapling system, in accordance with the present disclosure;

FIG. 5 illustrates graphs for exemplary firing strokes of a firing driver, in accordance with of the present disclosure;

FIG. 6 illustrates a circuit that includes capacitors in-line with field effect transistors, in accordance with the present disclosure;

FIG. 7 illustrates a switching capacitor circuit and graphs associated therewith, in accordance with the present disclosure;

FIG. 8A-8D illustrate voltage drops of power sources with different battery materials over time;

FIG. 9 illustrates graphs for an exemplary firing stroke of a firing driver, in accordance with the present disclosure;

FIG. 10 illustrates a method for controlling a surgical system, in accordance with the present disclosure;

FIG. 11 illustrates a battery, in accordance with the present disclosure;

FIG. 12 illustrates graphs for an exemplary firing stroke of a firing driver using the battery of FIG. 11, in accordance with the present disclosure;

FIG. 13 illustrates the load curve profile for a 15 W power supply;

FIG. 14 illustrates a circuit, in accordance with the present disclosure;

FIG. 15 illustrates a graph that illustrates button position of a surgical system, a switch position, and firing status of a motor over time, in accordance with the present disclosure;

FIG. 16 illustrates a method for controlling a surgical system, in accordance with the present disclosure;

FIG. 17 illustrates a method for controlling a surgical system, in accordance with the present disclosure;

Corresponding reference characters indicate corresponding parts throughout the several views.

### DESCRIPTION

Applicant of the present application owns the following U.S. patent applications that were filed on even date herewith and which are each herein incorporated by reference in their respective entireties:
U.S. patent application, titled METHOD OF OPERATING A SURGICAL STAPLING INSTRUMENT; Attorney Docket No. END9484USNP2/220491-2M;
U.S. patent application, titled SURGICAL STAPLING SYSTEMS WITH ADAPTIVE STAPLE FIRING ALGORITHMS; Attorney Docket No. END9484USNP3/220491-3;
U.S. patent application, titled LEARNED TRIGGERS FOR ADAPTIVE CONTROL OF SURGICAL STAPLING SYSTEMS; Attorney Docket No. END9484USNP4/220491-4;
U.S. patent application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. patent application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. patent application, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. patent application, titled SURGICAL TOOL WITH RELAXED FLEX CIRCUIT ARTICULATION; Attorney Docket No. END9484USNP9/220491-9;
U.S. patent application, titled WIRING HARNESS FOR SMART STAPLER WITH MULTI AXIS ARTICULATION; Attorney Docket No. END9484USNP10/220491-10;
U.S. patent application, titled SURGICAL SYSTEM WITH WIRELESS ARRAY FOR POWER AND DATA TRANSFER; Attorney Docket No. END9484USNP11/220491-11; and
U.S. patent application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. patent applications that were filed on even date herewith and which are each herein incorporated by reference in their respective entireties:
U.S. patent application, titled METHOD OF ASSEMBLING A STAPLE CARTRIDGE; Attorney Docket No. END9484USNP13/220491-13M;
U.S. patent application, titled CONTROL SURFACES ON A STAPLE DRIVER OF A SURGICAL STAPLE CARTRIDGE; Attorney Docket No. END9484USNP14/220491-14;
U.S. patent application, titled INTEGRAL CARTRIDGE STIFFENING FEATURES TO REDUCE CARTRIDGE DEFLECTION; Attorney Docket No. END9484USNP15/220491-15;
U.S. patent application, titled STAPLE CARTRIDGE COMPRISING WALL STRUCTURES TO REDUCE CARTRIDGE DEFLECTION; Attorney Docket No. END9484USNP16/220491-16;
U.S. patent application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. patent application, titled STAPLE CARTRIDGE COMPRISING A SLED HAVING A DRIVER LIFT CAM; Attorney Docket No. END9484USNP18/220491-18;
U.S. patent application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. patent application, titled STAPLE CARTRIDGE COMPRISING A COMPOSITE SLED; Attorney Docket No. END9484USNP20/220491-20;
U.S. patent application, titled SURGICAL INSTRUMENTS WITH JAW AND FIRING ACTUATOR LOCKOUT ARRANGEMENTS LOCATED PROXIMAL TO A JAW PIVOT LOCATION; Attorney Docket No. END9484USNP21/220491-21;
U.S. patent application, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22;
U.S. patent application, titled DUAL INDEPENDENT KEYED LOCKING MEMBERS ACTING ON THE SAME DRIVE MEMBER; Attorney Docket No. END9484USNP23/220491-23;
U.S. patent application, titled ADJUNCTS FOR USE WITH SURGICAL STAPLING INSTRUMENTS; Attorney Docket No. END9484USNP24/220491-24;
U.S. patent application, titled ADJUNCTS FOR USE WITH SURGICAL STAPLING INSTRUMENTS; Attorney Docket No. END9484USNP25/220491-25;
U.S. patent application, titled JAW CONTROL SURFACES ON A SURGICAL INSTRUMENT JAW; Attorney Docket No. END9484USNP26/220491-26;
U.S. patent application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. patent application, titled STAPLE CARTRIDGES COMPRISING TRACE RETENTION FEATURES; Attorney Docket No. END9484USNP29/220491-29; and
U.S. patent application, titled STAPLE CARTRIDGES COMPRISING STAPLE RETENTION FEATURES; Attorney Docket No. END9484USNP30/220491-30.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the described and illustrated embodiments are non-limiting examples, and thus it can be appreciated that the specific structural and functional details disclosed herein may be representative and illustrative. Variations and changes may be made without departing from the scope of the claims.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working frame through which the end effector and elongate shaft of a surgical instrument can be advanced.

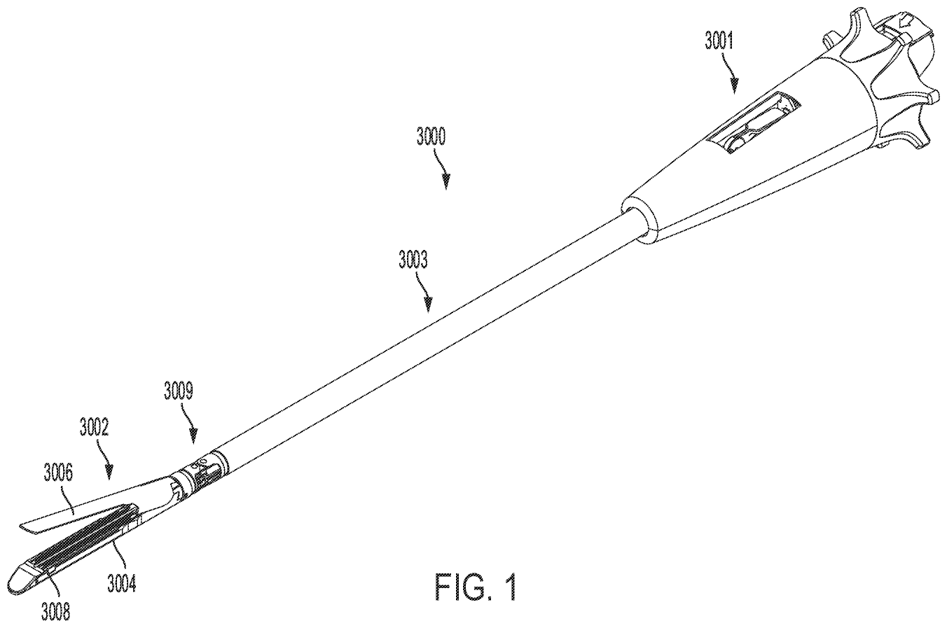

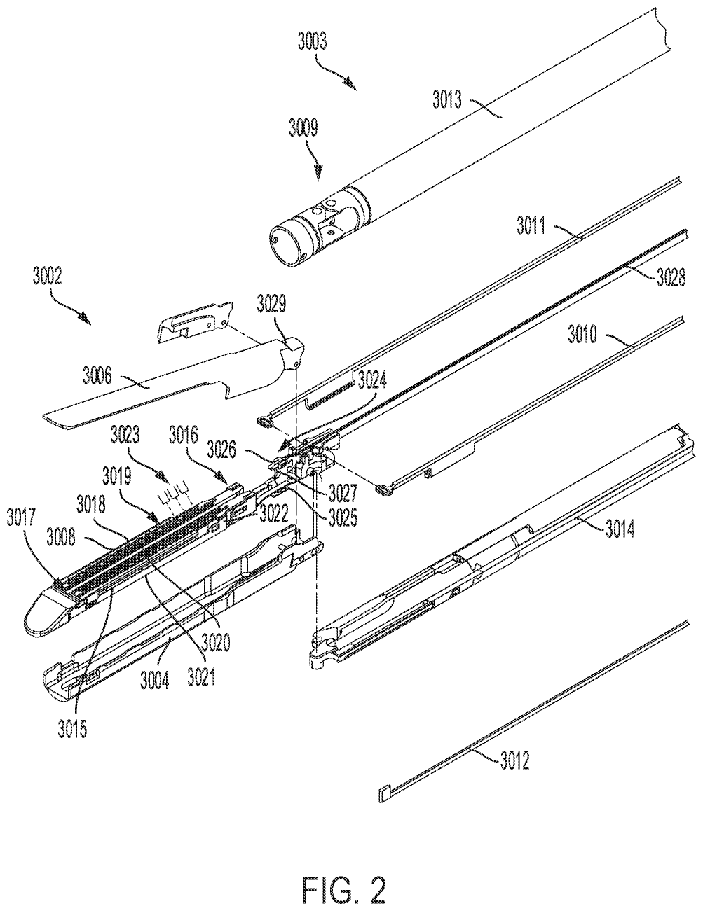

FIGS. 1 and 2 illustrate a surgical stapling system comprising a shaft assembly 3000 and an end effector 3002 extending from the shaft assembly 3000. The shaft assembly 3000 comprises an attachment portion 3001 and a shaft 3003 extending distally from the attachment portion 3001. The attachment portion 3001 is configured to be attached to a handle of a surgical instrument and/or the arm of a surgical robot, for example.

The end effector 3002 comprises a first jaw 3004 and a second jaw 3006. The first jaw 3004 comprises a staple cartridge 3008 insertable into and removable from the first jaw 3004; however, other embodiments are envisioned in which a staple cartridge is not removable from, or at least readily replaceable from, the first jaw 3004. The second jaw 3006 comprises an anvil configured to deform staples ejected from the staple cartridge 3008. The second jaw 3006 is pivotably coupled to the first jaw 3004 such that the second jaw 3006 is pivotable relative to the first jaw 3004 between an open position, where the tip of the second jaw 3006 is space apart from the first jaw 3004 (see FIG. 1) and a closed position, where the tip of the second jaw 3006 is adjacent the first jaw 3004 to capture tissue between the first jaw 3004 and the second jaw 3006; however, other embodiments are envisioned in which the first jaw 3004 is pivotable relative to the second jaw 3006.

The surgical stapling system comprises an articulation joint 3009 configured to permit the end effector 3002 to be rotated, or articulated, relative to the shaft 3003. The end effector 3002 is rotatable about an articulation axis extending through the articulation joint. Some embodiments may omit the articulation joint 3009. The shaft assembly 3000 comprises cooperating articulation rods 3010, 3011 configured to articulate the end effector 3002 relative to the shaft 3003 about the articulation joint 3009. The shaft assembly 3000 comprises an articulation lock bar 3012 configured to prevent rotation of the end effector 3002, an outer shaft tube 3013 configured to house internal components of the shaft assembly 3000, and a spine portion 3014 configured to provide structure support to the shaft assembly 3000.

The staple cartridge 3008 comprises a cartridge body 3015 including a deck 3018 extending between a proximal end 3016 and a distal end 3017. In use, the staple cartridge 3008 is positioned on a first side of tissue to be stapled and the anvil 3006 is positioned on a second side of the tissue. The anvil 3006 is moved toward the staple cartridge 3008 to compress and clamp the tissue against the deck 3018. Thereafter, staples 3023 removably stored in the cartridge body 3015 are deployed into the tissue. The cartridge body 3015 comprises a plurality of staples removably stored in a plurality of staple cavities 3019 defined within the cartridge body 3015. The staple cavities 3019 are arranged in six longitudinal rows. Three rows of staple cavities are positioned on a first side of a longitudinal slot 3020 and three rows of staple cavities are positioned on a second side of the longitudinal slot 3020. Other arrangements of staple cavities 3019 and staples may be possible.

The staples 3023 are supported by staple drivers in the cartridge body 3015. Staples supported on staple drivers are disclosed in U.S. Patent Application Publication No. 2021/0059672, which is herein incorporated by reference in its entirety. The drivers are movable between a first, unfired position, and a second, fired, position to eject the staples from the staple cavities 3019. The drivers are retained in the cartridge body 3015 by a retainer 3021 which extends around the bottom of the cartridge body 3015 and includes resilient members 3022 configured to grip the cartridge body 3015 and hold the retainer 3021 to the cartridge body 3015. The drivers are movable between their unfired positions and their fired positions by a sled. The sled is movable between a proximal position adjacent the proximal end 3016 and a distal position adjacent the distal end 3017. The sled comprises a plurality of ramped surfaces configured to slide under the drivers and lift the drivers, and the staples supported thereon, toward the anvil. In accordance with the present disclosure, the staples may not be supported by staple drivers, but rather, the staples may include integral drive surfaces that are directly engaged by the sled to lift the staples, examples of which are described in U.S. Patent Application Publication No. 2015/0173756, which is herein incorporated by reference in its entirety.

The sled is moved distally by a firing driver exemplified as a firing bar 3024 configured to contact the sled and push the sled toward the distal end 3017. The longitudinal slot 3020 defined in the cartridge body 3015 is configured to receive the firing driver 3024. The anvil 3006 also includes a slot configured to receive the firing driver 3024. The firing driver 3024 comprises a first cam 3025 which engages the first jaw 3004 and a second cam 3026 which engages the second jaw 3006. As the firing driver 3024 is advanced distally, the first cam 3025 and the second cam 3026 can control the distance, or tissue gap, between the deck 3018 of the staple cartridge 3008 and the anvil 3006. The firing driver 3024 also comprises a knife 3027 configured to incise the tissue captured intermediate the staple cartridge 3008 and the anvil 3006. The knife 3027 is desirably positioned at least partially proximal to the ramped surfaces to eject the staples ahead of the knife 3027. The shaft assembly 3000 comprises a firing bar 3028 attached to the firing driver 3024 and is configured to drive the firing driver through the staple cartridge 3008. In accordance with the present disclosure, the firing bar 3028 may comprise a plurality of laminated strips. More details of the shaft assembly 3000 are disclosed in U.S. patent application Ser. No. 15/385,887 entitled METHOD FOR ATTACHING A SHAFT ASSEMBLY TO A SURGICAL INSTRUMENT AND, ALTERNATIVELY, TO A SURGICAL ROBOT, which is herein incorporated by reference in its entirety.

In accordance with the present disclosure, the anvil 3006 may be moved from the open position to the closed position using a closure system that is controlled separately from the firing driver 3024, where the firing driver 3024 is considered to be a part of a firing system that is separate and distinctly operable from the closure system. Further, in accordance with the present disclosure, the anvil 3006 may comprise a ramp 3029 on a proximal end thereof and the closure system may comprise a closure member, such as an outer shaft tube 3013, that can be movable distally to engage the ramp 3029 and cam the anvil 3006 to the closed position. In the closed position, the first cam 3025 and the second cam 3026 of the firing driver 3024 translate distally and maintain the anvil 3006 in the closed position. To transition the anvil 3006 to the open position, the closure member may be retracted proximal and the anvil 3006 may be biased to the open position by springs positioned within the end effector 3002. The anvil 3006 may include a tab and the closure member may define an aperture at the distal end thereof which engages the tab as the closure member moves proximally, thereby positively transitioning the anvil 3006 to the open position. Exemplary closure systems and closure members are disclosed in U.S. Patent Application Publication No. 2021/0059672, the entire disclosure of which is hereby incorporated by reference herein.

In accordance with the present disclosure, the firing driver 3024 may move the anvil 3006 from the open position to the closed position. The anvil 3006 includes a ramp that extends from a wall defining the slot in the anvil 3006 and that is engaged by the firing driver 3024 during a first portion of the stroke of the firing driver 3024 to move the anvil 3006 to the closed position. At the end of the first portion of the stroke, the firing driver 3024 can continue advancing distally through a second portion of the stroke to deploy staples from the staple cartridge 3008 and incise tissue captured by the end effector 3002. Exemplary firing drivers that close the anvil and fire staples are disclosed in U.S. Pat. No. 11,160,551.

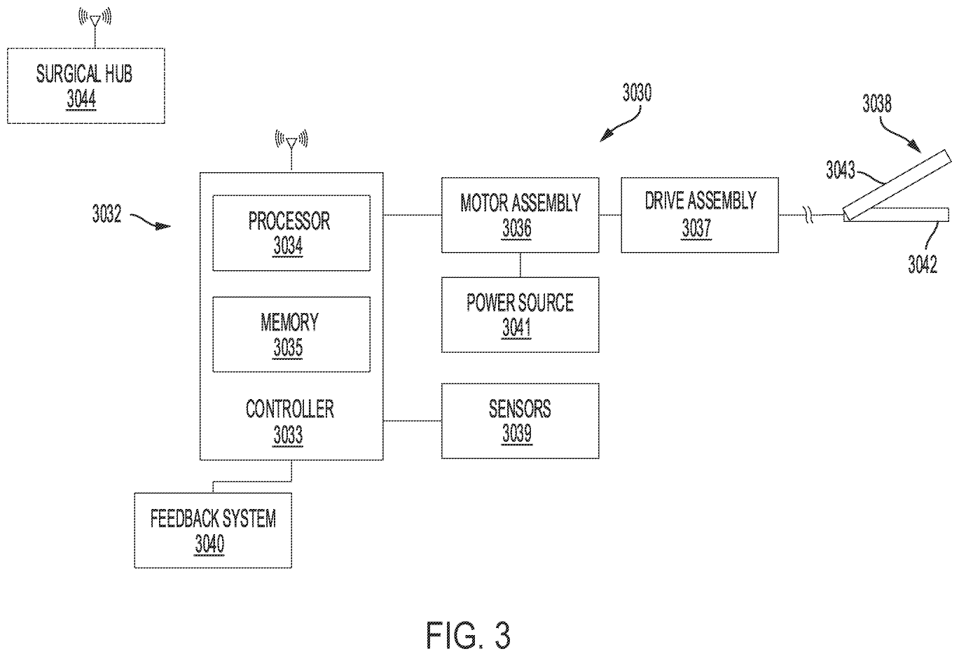

FIG. 3 illustrates a surgical system 3030 for use with one or more surgical instruments, tools, and/or robotic systems in accordance with the present disclosure. The surgical system 3030 includes a control circuit 3032. The control circuit 3032 includes a microcontroller 3033 comprising a processor 3034 and a storage medium such as, for example, a memory 3035.

A motor assembly 3036 includes a motor, driven by a motor driver. The motor assembly 3036 operably couples to a drive assembly 3037 to drive, or effect, motion at an end effector 3038, similar to the end effector 3002 shown in FIGS. 1 and 2. The drive assembly 3037 may include any number of components suitable for transmitting motion to the end effector 3038 such as, for example, one or more linkages, bars, tubes, and/or cables, for example. In accordance with the present disclosure, the drive assembly 3037 can drive a firing driver and/or a closure member.

A sensor(s) 3039, for example, provides real-time feedback to the processor 3034 about an operational parameter monitored during a surgical procedure being performed by the surgical system 3030. The operational parameter can be associated with a user performing the surgical procedure, a tissue being treated, and/or one or more components of the surgical system 3030, for example. The sensor 3039 may comprise one or more than one suitable sensor, such as, for example, a magnetic sensor, such as a Hall effect sensor, a strain gauge, a pressure sensor, a force sensor, an inductive sensor, such as an eddy current sensor, a resistive sensor, a capacitive sensor, an optical sensor, a current sensor, a voltage sensor, and/or any other suitable sensor.

The sensor(s) 3039 may comprise one or more than one suitable sensor for detecting one or more than one condition at the end effector 3038 including, without limitation, a tissue thickness sensor such as a Hall Effect Sensor or a reed switch sensor, an optical sensor, a magneto-inductive sensor, a force sensor, a pressure sensor, a piezo-resistive film sensor, an ultrasonic sensor, an eddy current sensor, an accelerometer, a pulse oximetry sensor, a temperature sensor, a sensor configured to detect an electrical characteristic of a tissue path (such as capacitance or resistance), or any combination thereof. As another example, and without limitation, the sensor(s) 3039 may include a sensor located at, or about, an articulation joint, similar to articulation joint 3009, extending proximally from the end effector 3038. Such sensors may include, for example, a potentiometer, a capacitive sensor (slide potentiometer), piezo-resistive film sensor, a pressure sensor, a pressure sensor, or any other suitable sensor type. In accordance with the present disclosure, the sensor(s) 3039 may comprise a plurality of sensors located in multiple locations in the end effector 3038.

In accordance with the present disclosure, the system 3030 may include a feedback system 3040 which may include a device for providing sensory feedback to a user. Such devices may comprise, for example, visual feedback devices (e.g., an LCD display screen, a touch screen, LED indicators), audio feedback devices (e.g., a speaker, a buzzer) or tactile feedback devices (e.g., haptic actuators).

The microcontroller 3033 may be programmed to perform various functions such as precise control over the speed and position of the drive assembly 3037. In accordance with the present disclosure, the microcontroller 3033 may be any single-core or multicore processor such as those known under the trade name ARM Cortex by Texas Instruments. Further, in accordance with the present disclosure, the main microcontroller 1933 may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

The microcontroller 3033 may be configured to compute a response in the software of the microcontroller 3033. The computed response is compared to a measured response of the actual system to obtain an “observed” response, which is used for actual feedback decisions. The observed response is a favorable, tuned value that balances the smooth, continuous nature of the simulated response with the measured response, which can detect outside influences on the system.

The motor assembly 3036 includes one or more than one electric motor and one or more than one motor driver. The electric motor may be a brushed direct current (DC) motor with a gearbox and mechanical links to the drive assembly 3037. In accordance with the present disclosure, a motor driver may be an A3941 available from Allegro Microsystems, Inc.

In various forms, the motor assembly 3036 includes a brushed DC driving motor having a maximum rotational speed of approximately 25,000 RPM. The motor assembly 3036 may include a brushless motor, a cordless motor, a synchronous motor, a stepper motor, or any other suitable electric motor. The motor driver may comprise an H-bridge driver comprising field-effect transistors (FETs), for example. Those skilled in the art will appreciate that an amount of power any motor produces is determined solely by the voltage applied to the motor and the current drawn by the windings of the motor.

The motor assembly 3036 can be powered by a power source 3041. In accordance with the present disclosure, the power source 3041 may include one or more than one battery to power the motor assembly 3036. A battery may include a number of battery cells connected in series. The battery cells may be replaceable and/or rechargeable. Additionally, or alternatively, the battery cells can be lithium-ion batteries coupleable to and separable from the power assembly.

The end effector 3038 includes a first jaw 3042 and a second jaw 3043. At least one of the first jaw 3042 or the second jaw 3043 is rotatable relative to the other during a closure motion that transitions the end effector 3038 from an open configuration to a closed configuration. The closure motion may cause the jaws 3042, 3043 to grasp tissue therebetween. In accordance with the present disclosure, sensors, such as, for example, a strain gauge or a micro-strain gauge, can be configured to measure a parameter of the end effector 3038, such as, for example, the amplitude of the strain exerted on one or both of the jaws 3042, 3043 during a closure motion, which can be indicative of the closure forces applied to the jaws 3042, 3043. The measured strain is converted to a digital signal and provided to the processor 3034, for example. Alternatively or additionally, sensors such as, for example, a load sensor, can measure a closure force and/or a firing force applied to the jaws 3042, 3043. In accordance with the present disclosure, the sensors may comprise a first sensor to measure a first force on a firing driver 3024 during a firing stroke, and a second sensor to measure a second force on a closure member, such as the outer shaft tube 3013, during a closure stroke. The processor 3034 can receive these force measurements and determine a relationship therebetween, such as a distribution ratio of the force exerted on the firing driver 3024 and the outer shaft tube 3013.

In accordance with the present disclosure, a current sensor can be employed to measure the current drawn by a motor of the motor assembly 3036. The force required to advance the drive assembly 3037 can correspond to the current drawn by the motor, for example. The measured force is converted to a digital signal and provided to the processor 3034.

In accordance with the present disclosure, a strain gauge sensor can measure the force applied to the tissue by the end effector 3038. The strain gauge sensor can be coupled to the end effector 3038 to measure the force on the tissue being treated by the end effector 3038. The strain gauge sensor can measure the amplitude or magnitude of the strain exerted on a jaw of an end effector 3038 during a closure motion, which can be indicative of the tissue compression. The measured strain is converted to a digital signal and provided to a processor 3034.

The measurements of the tissue compression, tissue thickness, and/or force required to close the end effector on the tissue, as respectively measured by the sensors 3039 can be used by the microcontroller 3033 to characterize the selected position and/or corresponding value of the speed of one or more than one component of the drive assembly 3037. In accordance with the present disclosure, a memory 3035 can store instructions, an equation, and/or a lookup table which can be employed by the microcontroller 3033 in the assessment of position and speed on the one or more than one component of the drive assembly 3037.

The surgical system 3030 may comprise wired or wireless communication circuits to communicate with surgical hubs (e.g., surgical hub 3044), communication hubs, and/or robotic surgical hubs, for example. Additional details about suitable interactions between a surgical system 3030 and the surgical hub 3044 are disclosed in U.S. patent application Ser. No. 16/209,423 entitled METHOD OF COMPRESSING TISSUE WITHIN A STAPLING DEVICE AND SIMULTANEOUSLY DISPLAYING THE LOCATION OF THE TISSUE WITHIN THE JAWS, now U.S. Patent Application Publication No. 2019/0200981, the entire disclosure of which is herein incorporated by reference in its entirety.

The control circuit 3032 can be configured to implement various processes described herein. In accordance with the present disclosure, the control circuit 3032 may comprise a microcontroller 3033 comprising processor 3034 (e.g., microprocessor) coupled to a memory circuit 3035. The memory circuit 3035 stores machine-executable instructions that, when executed by the processor 3034, cause the processor 3034 to execute machine instructions to implement various processes described herein. The processor 3034 may be a single-core or multicore processor. The memory circuit 3035 may comprise volatile or non-volatile storage media. The processor 3034 may include a central processing unit (CPU) and an arithmetic unit. The CPU may be configured to receive instructions from the memory circuit 3035.

Alternatively, the control circuit 3032 can be a combinational logic circuit configured to implement various processes described herein. The combinational logic circuit may comprise a finite state machine comprising a combinational logic configured to receive data, process the data by the combinational logic, and provide an output.

Alternatively, the control circuit 3032 is a sequential logic circuit configured to implement various processes described herein. The sequential logic circuit may comprise a finite state machine. The sequential logic circuit may comprise a combinational logic, at least one memory circuit, and a clock, for example. The at least one memory circuit can store a current state of the finite state machine. In accordance with the present disclosure, the sequential logic circuit may be synchronous or asynchronous. The control circuit 3032 may comprise a combination of a processor (e.g., processor 3034) and a finite state machine to implement various processes herein. The finite state machine may comprise a combination of a combinational logic circuit (and the sequential logic circuit, for example.

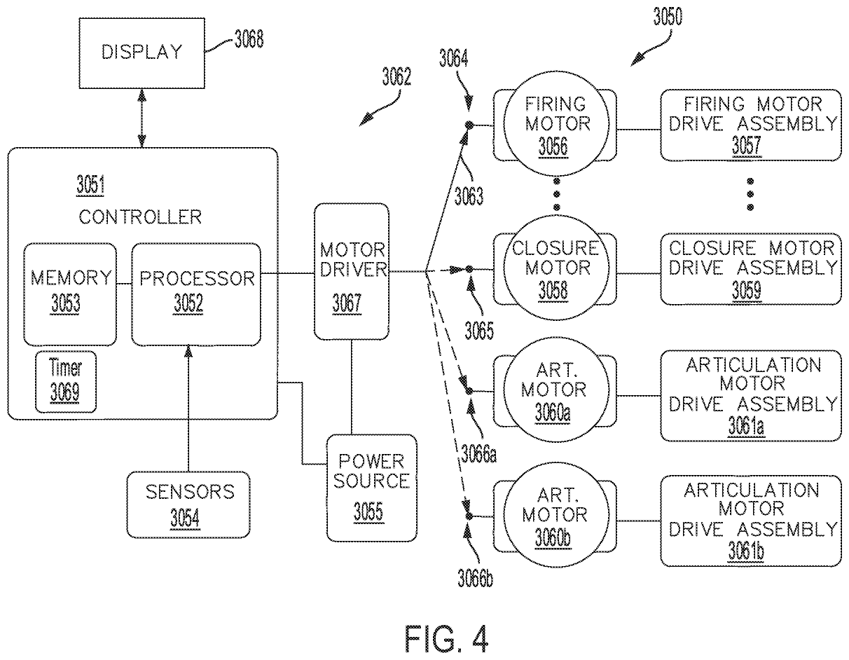

FIG. 4 is a block diagram of a surgical system 3050 for use with one or more surgical instruments, tools, and/or robotic systems in accordance with the present disclosure. The surgical system 3050 is similar in many respects to the surgical system 3030, which are not repeated herein at the same level of detail for brevity. For example, like the surgical system 3030, the surgical system 3050 includes a control circuit comprising a microcontroller 3051 comprising a processor 3052 and a memory 3053, a sensor 3054, and a power source 3055, which are similar, respectively, to the microcontroller 3033, the processor 3034, the memory 3035, and the power source 3041. Additionally, the surgical system 3050 includes a plurality of motors and corresponding driving assemblies that can be activated to perform various functions.

In accordance with the present disclosure, a first motor can be activated to perform a first function, a second motor can be activated to perform a second function, a third motor can be activated to perform a third function, a fourth motor can be activated to perform a fourth function, and so on. Additionally, in accordance with the present disclosure, the plurality of motors can be individually activated to cause firing, closure, and/or articulation motions in an end effector, such as end effector 3002 or end effector 3038, as examples. The firing, closure, and/or articulation motions can be transmitted to the end effector through a shaft assembly, such as shaft assembly 3000, for example.

The surgical system 3050 may include a firing motor 3056 operably coupled to a firing motor drive assembly 3057, which can be configured to transmit firing motions, generated by the motor 3056, to the end effector The firing motions including, for example, displacement of the firing bar 3028 and firing driver 3024. The firing motions generated by the motor 3056 may deploy the staples from the staple cartridge 3008 into tissue captured by the end effector, and/or advance the firing driver the knife 3027 to cut the captured tissue. The firing driver may be retracted by reversing the direction of the motor 3056.

The surgical system 3050 may include a closure motor 3058 operably coupled to a closure motor drive assembly 3059 configured to transmit closure motions, generated by the motor 3058, to the end effector. In particular, the closure motions displace a closure member, such as outer shaft tube, to close an anvil and compress tissue between the anvil and the staple cartridge. The closure motions may cause the end effector to transition from an open configuration to an approximated, or closed, configuration to grasp tissue, for example. The end effector may be transitioned to an open position by reversing the direction of the motor 3058.

The surgical system 3050 may include one or more than one articulation motors 3060a, 3060b, operably coupled to respective one or more than one articulation motor drive assemblies 3061a, 3061b configured to transmit articulation motions, generated by the motors 3060a, 3060b, to the end effector. The articulation motions may cause the end effector to articulate relative to a shaft, for example. In accordance with the present disclosure, the first articulation motor 3060a may drive a first articulation bar, such as articulation rod 3010, to rotate the end effector in a first direction and the second articulation motor 3060b may drive a second articulation bar, such as articulation bar 3011, to rotate the end effector in a second direction opposite the first direction.

The surgical system 3050 may include a plurality of motors configured to perform various independent functions. In accordance with the present disclosure, the plurality of motors of the surgical instrument or tool can be individually or separately activated to perform one or more than one function while the other motors remain inactive. The articulation motors 3060a, 3060b can be activated to cause the end effector to be articulated while the firing motor 3056 remains inactive. Alternatively, the firing motor 3056 can be activated to fire the plurality of staples, and/or to advance the cutting edge, while the articulation motors 3060a, 3060b remains inactive. The closure motor 3058 may be activated simultaneously with the firing motor 3056 to cause the closure member and the firing driver to advance distally at the same time, or in an overlapping fashion, as described in more detail herein below.

The surgical system 3050 may include a common control module 3062 which can be employed with a plurality of motors of the surgical instrument or tool. The common control module 3062 may accommodate one of the plurality of motors at a time. For example, the common control module 3062 can be coupleable to and separable from the plurality of motors of the robotic surgical instrument individually. A plurality of the motors of the surgical instrument or tool may share the common control module 3062. A plurality of motors of the surgical instrument or tool can be individually and selectively engaged with the common control module 3062. The common control module 3062 can be selectively switched from interfacing with one of a plurality of motors of the surgical instrument or tool to interfacing with another one of the plurality of motors of the surgical instrument or tool.

The common control module 3062 can be selectively switched between operable engagement with the articulation motors 3060a, 3060b and operable engagement with either the firing motor 3056 or the closure motor 3058. In the example illustrated in FIG. 4, a switch 3063 can be moved or transitioned between a plurality of positions and/or states. In a first position 3064, the switch 3063 may electrically couple the common control module 3062 to the firing motor 3056; in a second position 3065, the switch 3063 may electrically couple the common control module 3062 to the closure motor 3058; in a third position 3066a, the switch 3063 may electrically couple the common control module 3062 to the first articulation motor 3060a; and in a fourth position 3066b, the switch 3063 may electrically couple the common control module 3062 to the second articulation motor 3060b, for example. Separate common control modules 3062 can be electrically coupled to the firing motor 3056, the closure motor 3058, and the articulation motor 3060a, 3060b at the same time. The switch 3063 may be a mechanical switch, an electromechanical switch, a solid-state switch, or any suitable switch.

Each of the motors 3056, 3058, 3060a, 3060b may comprise a torque sensor to measure the output torque on the shaft of the motor. The force on an end effector may be sensed in any conventional manner, such as by force sensors on the outer sides of the jaws or by a torque sensor for the motor actuating the jaws.

As illustrated in FIG. 4, the common control module 3062 may comprise a motor driver 3067, which may comprise one or more H-Bridge FETs. The motor driver 3067 may modulate the power transmitted from a power source 3055 to a motor coupled to the common control module 3062 based on input from a microcontroller 3051 (the “controller”), for example. In accordance with the present disclosure, the microcontroller 3051 can be employed to determine the current drawn by the motor, for example, while the motor is coupled to the common control module 3062.

The processor 3052 may control the motor driver 3067 to control the position, direction of rotation, and/or velocity of a motor that is coupled to the common control module 3062. In accordance with the present disclosure, the processor 3052 can signal the motor driver 3067 to stop and/or disable a motor that is coupled to the common control module 3062.

The memory 3053 may include program instructions for controlling each of the motors of the surgical system 3050 coupleable to the common control module 3062. For example, the memory 3053 may include program instructions for controlling the firing motor 3056, the closure motor 3058, and the articulation motors 3060a, 3060b to cause the processor 3052 to control the firing, closure, and articulation functions in accordance with inputs from algorithms or control programs of the surgical instrument or tool.

A mechanism and/or sensor(s) 3054 can be employed to alert the processor 3052 to the program instructions that should be used in a particular setting. For example, the sensor(s) 3054 may alert the processor 3052 to use the program instructions associated with firing, closing, and articulating the end effector. In accordance with the present disclosure, the sensor(s) 3054 may comprise position sensors to sense the position of the switch 3063, for example. Accordingly, the processor 3052 may use the program instructions associated with firing the I-beam of the end effector upon detecting, through the sensors 3054 for example, that the switch 3063 is in the first position 3064; the processor 3052 may use the program instructions associated with closing the anvil upon detecting, through the sensors 3054 for example, that the switch 3063 is in the second position 3065; and the processor 3052 may use the program instructions associated with articulating the end effector upon detecting, through the sensors 3054 for example, that the switch 3063 is in the third or fourth position 3066a, 3066b. In accordance with the present disclosure, the controller 3051 can communicate with a display 3068, which can be similar to feedback system 3040, to provide feedback to a user. In addition, the display 3068 can include an input interface such that a user can provide input for controlling the surgical system 3050. The controller 3051 can include a timer 3069 to measure elapsed time.

Referring also to FIGS. 1 and 2, a surgical system can include a closure system and a firing system. The closure system includes a closure member, such as the outer shaft tube 3013, that is movable from a proximal position toward a distal position during a closure stroke. The closure member is driven between the proximal position and the distal position by the closure motor 3058; however, other embodiments are envisioned where the closure member is driven between the proximal position and the distal position by a manual-drive system that includes a closure trigger manually operable by a clinician. The surgical system comprises an end effector 3002 comprising a first jaw 3004, and a second jaw 3006, that is rotatable relative to the first jaw between an open position and a closed position. In use, the closure member translates toward the distal position and engages the second jaw, such as on a ramp on the proximal end thereof. The closure member applies a closure force to the second jaw to rotate the second jaw toward the closed position. In accordance with the present disclosure, the surgical system may include a power source 3041, 3055, to power the closure motor.

The firing system includes a firing driver 3024, that is movable from a proximal, unfired position, toward a distal, fired position, during a firing stroke to deploy staples stored in a staple cartridge 3008, and to incise tissue captured by the end effector with a knife 3027. The firing driver is driven between the proximal, unfired position and the distal, fired position by a firing motor 3056. The firing driver includes a first cam 3025 and a second cam 3026 to engage the first jaw and the second jaw, respectively, during the firing stroke to apply a closure force to the end effector to maintain the second jaw in the closed position. In accordance with the present disclosure, a power source 3041, 3055 may power the firing motor.

The surgical system includes a control system 3033 or controller 3051, as examples, to actuate the closure motor and firing motor to drive the closure member and the firing driver, respectively, through their respective strokes. The surgical system includes a voltage sensor 3039, to sense a voltage potential of the power source.

During use, the closure motor and firing motor draw current and consume power from the power source to drive the closure member and firing driver, respectively, through their respective strokes. As the motor draws current and consume power from the power source, the voltage potential of the power source is loaded and the voltage drops, or sags, causing the power output of the motors to drop. It is desirable to minimize power source voltage drop over the closure stroke/firing stroke of the closure member/firing driver, respectively, to increase the power output of the power source to the respective motors over their respective strokes. It is desirable to maximize the short inconsistent current draw from the power source by the motor during use thereof.

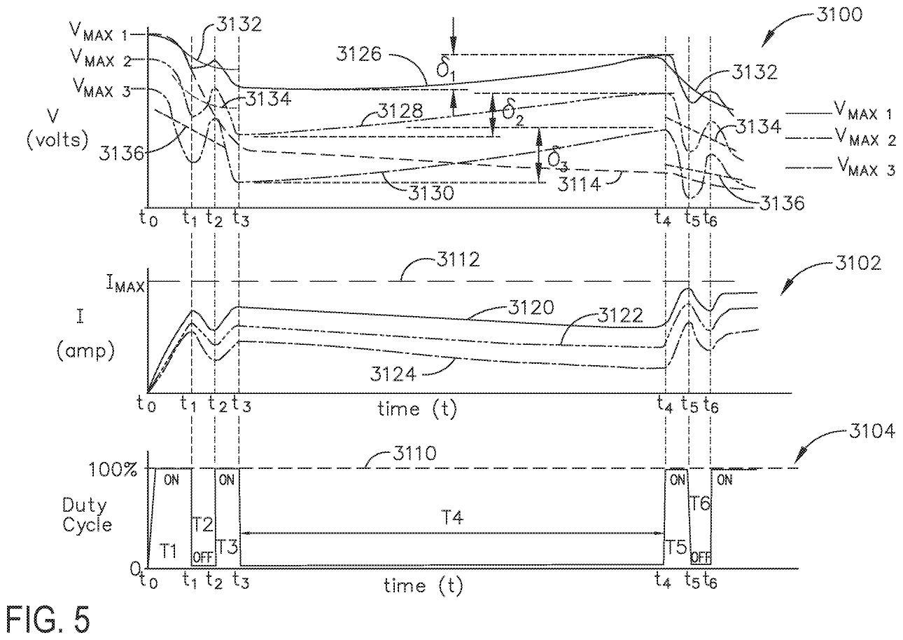

Voltage drop can be minimized by way of pulse width modulation and micro-recoveries during each “off” period of the pulses. Referring now to FIG. 5, graphs 3100, 3102, 3104 illustrate exemplary firing strokes of a firing driver through a staple cartridge. Graph 3100 illustrates the voltage potentials of power sources over time. Graph 3102 illustrates the currents sourced by the respective power sources over time. Graph 3104 illustrates the duty cycle of the respective power sources over time.

In accordance with the present disclosure, the control system may implement a firing algorithm, which may cause a firing motor 3056, to draw current from a power source having a maximum (or peak) voltage potential V_{MAX1} and drive a firing driver 3024 through a firing stroke. The algorithm implements a duty cycle 3110 of 100% (e.g., where the motor is held in an “on” state), as shown in graph 3104. Based on the algorithm, the current 3112 drawn by the motor from the power source is held constant, or substantially constant, at I_{MAX} and the voltage potential 3114 of the power source drops from the maximum voltage potential V_{MAX1} to a minimum voltage potential at the end of the firing stroke.

In accordance with the present disclosure, the control system may implement an adaptive firing algorithm that causes the firing motor to drive the firing driver with adaptive pulse width modulation to diminish power source voltage drop over the firing stroke. Further, in accordance with the present disclosure, graphs 3100, 3102, 3104 may illustrate three exemplary firing strokes of a firing driver using the adaptive firing algorithm using three different power sources-a first power source having a maximum voltage potential of V_{MAX1}, a second power source having a voltage potential of V_{MAX2}, and a third power source having a voltage potential of V_{MAX3}. The first power source includes cells that each have a voltage potential of V_{cell1} that collectively form the first power source. The second power source includes cells that each have a voltage potential of V_{cell2} that collectively form the second power source. The third power source includes cells that each have a voltage potential of V_{cell3} that collectively form the third power source.

As shown in graphs 3100, 3102, 3104, at to, the control system implements the adaptive firing algorithms, which causes the firing motor to transition from an “off” state, in which the motor does not drive, or ceases to drive, the firing driver to prevent it from moving toward the fired position, to an “on” state, in which the motor drives the firing driver toward the fired position, for a first period T1. During the first period T1, the current 3120 drawn by the motor from the first power source increases from 0 to a first maximum current I_{MAX1} and the voltage potential 3126 applied to the motor from the first power source drops from the maximum voltage potential V_{MAX1} to a first lower voltage potential. Similarly, the current 3122 drawn by the motor from the second power source increases from 0 to a first max current I_{MAX2} and the voltage potential 3128 applied to the motor from the second power source drops from the maximum voltage potential V_{MAX2} to a first lower voltage potential. Similarly, the current 3124 applied to the motor from the third power source increases from 0 to a first max current I_{MAX3} and the voltage potential 3130 applied to the motor from the third power source drops from the maximum voltage potential V_{MAX3} to a first lower voltage potential. In accordance with the present disclosure, the control system can detect, or measure, the currents drawn by the motors and voltages applied to the motors using a current sensor and a voltage sensor, respectively. It will be appreciated that the motor can drive the firing driver in either a forward direction (proximal to distal) or a backward direction (distal to proximal).

In accordance with the present disclosure, T1 may be a predetermined period that may be stored in a memory 10035, and may be retrievable by the control system. Further, in accordance with the present disclosure, T1 may be based on the implemented duty cycle. Alternatively, or additionally, T1 may be a variable period. Alternatively, or additionally, T1 may be based on a rate at which the voltage potential drops from the maximum voltage potential. Alternatively, or additionally, T1 may be based on the maximum voltage potential dropping a predetermined amount. Alternatively, or additionally, T1 may be based on the maximum voltage potential dropping to a predetermined lower voltage potential.

At time t₁, the algorithm automatically causes the motor to transition to the “off” state for a second period T2, during which time the current 3122, 3122, 3124 drawn by the motor from the power sources drops from the first max current I_{MAX1} to a first lower current and the voltage potential 3126, 3128, 3130 of the power sources recovers from the first lower voltage potential to a first recovered voltage potential that is less than the maximum voltage potentials of the respective power sources.

In accordance with the present disclosure, T2 may be a predetermined period that may be stored in a memory 10035, and may be retrievable by the control system. Alternatively, or additionally, T2 may be based on the implemented duty cycle. Alternatively, or additionally, T2 may be a variable period. Alternatively, or additionally, T2 may be based on T1. Alternatively, or additionally, T2 may be based on a magnitude of the voltage potential drop over T1. Alternatively, or additionally, T2 may be based on a rate at which the voltage potential dropped over T1. Alternatively, or additionally, T2 may be based on the time required for the power source to recover a threshold amount of voltage potential from the first dropped voltage potential. Alternatively, or additionally, T2 may be based on a rate at which the voltage potential recovers from the first dropped voltage potential. In accordance with the present disclosure, T2 may be different than T1. Alternatively, or additionally, T2 may be the same as T1

At time t₂, the algorithm automatically causes the motor to transition to the “on” state for a third period T3, during which time the current 3122, 3122, 3124 drawn by the motor from the power sources increases from the first lower current to a second maximum current that is greater than the first maximum current I_{MAX1} and the voltage potential 3126, 3128, 3130 applied to the motor from the power sources drops from the first recovered voltage potential to a second lower voltage potential that is less than the first lower voltage potential.

In accordance with the present disclosure, T3 may be a period stored in a memory 10035, and may be retrievable by the control system. Alternatively, or additionally, T3 may be based on the implemented duty cycle. In accordance with the present disclosure, T3 may be a variable period. Alternatively, or additionally, T3 may be based on T1 or T2, or a combination thereof. Alternatively, or additionally, T3 may be based on a rate at which the voltage potential recovered during T2. Alternatively, or additionally, T3 may be based on a magnitude of the voltage potential recovered during T2. Alternatively, or additionally, T3 may be based on a rate at which the voltage drops from the first recovered voltage potential. Alternatively, or additionally, T3 may be based on the voltage potential dropping a predetermined amount from the first recovered voltage potential. Alternatively, or additionally, T3 may be based on the voltage potential dropping to a predetermined voltage potential from the first recovered voltage potential. Alternatively, or additionally, T3 may be different than T1 and/or T2. Alternatively, or additionally, T3 may be the same as T1 and/or T3.

At time t₃, the algorithm automatically causes the motor to transition to the “off” state for a fourth period T4, during which time the current 3122, 3122, 3124 drawn by the motor from the power sources drops from the second maximum current I_{MAX2} to a second lower current that is less than the first lower current and the voltage potential 3126, 3128, 3130 of the power sources recovers from the second lower voltage potential to a second recovered voltage potential that is less than the first recovered voltage potential.

In accordance with the present disclosure, T4 may be a predetermined period that may be stored in a memory 10035, and may be retrievable by the control system. Alternatively, or additionally, T4 may be a variable period. Alternatively, or additionally, T4 may be based on T1, T2, or T3, or any combination thereof. Alternatively, or additionally, T4 may be based on a magnitude of the voltage potential drop over T1 or T3, or a combination thereof. Alternatively, or additionally, T4 may be based on a rate at which the voltage potential dropped over T1 or T3, or a combination thereof. Alternatively, or additionally, T4 may be based on the time required for the power source to recover a threshold amount of voltage potential from the second dropped voltage potential. Alternatively, or additionally, T4 may be based on a rate at which the voltage potential recovers from the second dropped voltage potential or a rate at which the voltage potential recovered from the first dropped voltage potential, or a combination thereof. Alternatively, or additionally, T4 may be different than T1, T2, and/or T3. Alternatively, or additionally, T4 may be the same as T1, T2 and/or T3. Alternatively, or additionally, T4 may be about 10 to 50 times larger than T2. As shown in graph 3100, during the fourth period T4, the motor is maintained in an “off” state for a period that allows the power sources to recover a δ amount. Specifically, the motor remains in the “off” state for the fourth period T4 such that the first power source recovers a first amount δ₁, the second power source recovers a second amount δ₂, and the third power source recovers a third amount δ₃.

At time t₄, the algorithm automatically causes the motor to transition to the “on” state for a fifth period T5, during which time the current 3122, 3122, 3124 drawn by the motor from the power sources increases from the second lower current to a third maximum current I_{MAX3} and the voltage potential 3126, 3128, 3130 applied to the motor from the power sources drops from the second recovered voltage potential to a third lower voltage potential.

In accordance with the present disclosure, T5 may be a predetermined period that is stored in a memory 10035, and is retrievable by the control system. Further, in accordance with the present disclosure, T5 may be based on the implemented duty cycle. Alternatively, or additionally, T5 may be a variable period. Additionally, in accordance with the present disclosure, T5 may be based on T1 T2, T3, or T4, or any combination thereof. Alternatively, or additionally, T5 may be based on a rate at which the voltage potential recovered during T2 or T4, or a combination thereof. Alternatively, or additionally, T5 may be based on a magnitude of the voltage potential recovered during T2 or T4, or a combination thereof. Alternatively, or additionally, T5 may be based on a rate at which the voltage drops from the second recovered voltage potential or a rate at which the first recovered voltage potential dropped, or a combination thereof. Alternatively, or additionally, T5 may be based on the voltage potential dropping a predetermined amount from the second recovered voltage potential. Alternatively, or additionally, T5 may be based on the voltage potential dropping to a predetermined voltage potential from the second recovered voltage potential. Alternatively, or additionally, T5 may be different than T1, T2, T3 and/or T4. Alternatively, or additionally, T5 may be the same as T1, T2, T3 and/or T4.

At time t₅, the algorithm automatically causes the motor to transition to the off state for a sixth period T6, during which time the current 3122, 3122, 3124 applied to the motor from the power sources drops from the third maximum current I_{MAX3} to a third lower current and the voltage potential 3126, 3128, 3130 of the power sources recovers from the third lower voltage potential to a third recovered voltage potential.

In accordance with the present disclosure, T6 may be a predetermined period that may be stored in a memory 10035, and may be retrievable by the control system. Alternatively, or additionally, T6 may be based on the implemented duty cycle. Alternatively, or additionally, T6 may be a variable period. Alternatively, or additionally, T6 may be based on T1, T2, or T3, T4, or T5, or any combination thereof. Alternatively, or additionally, T6 may be based on a magnitude of the voltage potential drop over T1, T3, or T5, or any combination thereof. Alternatively, or additionally, T6 may be based on a rate at which the voltage potential dropped over T1, T3, or T5, or any combination thereof. Alternatively, or additionally, T6 may be based on the time required for the power source to recover a threshold amount of voltage potential from the third dropped voltage potential. Alternatively, or additionally, T6 may be based on a rate at which the voltage potential recovers from the third lower voltage potential, a rate at which the voltage potential recovered from the first lower voltage potential, or a rate at which the voltage potential recovered from the second lower voltage potential, or any combination thereof. In accordance with the present disclosure, T6 may be different than T1, T2, T3, T4 and/or T5. Alternatively, or additionally, T6 is the same as T1, T2, T3, T4, and/or T5.

At time t₆, the algorithm automatically causes the motor to transition to the on state for a seventh period T7, during which time the current 3122, 3122, 3124 applied to the motor from the power sources increases from the third lower current and the voltage potential 3126, 3128, 3130 applied to the motor from the power source drops from the third recovered voltage potential. During the seventh period t7, the firing driver reaches the distal fired position of the firing stroke and the firing algorithm ceases.

As shown, the algorithm is adaptive and reacts to changes in the voltage potential over the firing stroke of the firing driver. Based on the adaptive nature of the algorithm and the “micro-recoveries” of the voltage potential during the firing stroke, the overall macro-recovery curves 3132, 3134, 3136 of the voltage potentials over time are substantially higher compared to when the motor is left “on” (duty cycle of 100%) for the entire firing stroke, or the firing algorithm uses a constant pulse width modulation algorithm. In addition, the algorithm implemented by the control system implements a variable length pulse width modulation that has variable on/off times based the voltage drops and recovers over the firing stroke.

The first power source, which has a first maximum voltage potential V_{MAX1}, experiences less modulation during the algorithm than the second power source, which has a second maximum voltage potential V_{MAX2} less than the first maximum voltage potential V_{MAX1}. Similarly, the second power source, which has a second maximum voltage potential V_{MAX2}, experiences less modulation during the algorithm than the third power source, which has a third maximum voltage potential V_{MAX3} less than the second maximum voltage potential V_{MAX2}. In accordance with the present disclosure, referring to graph 3100, during the second recovery period T4, the first power source recovers δ1, the second power source recovers δ2 which is more than 01, and the third power source recovers δ3 which is more than δ1 and δ3.

In accordance with the present disclosure, utilizing pulse width modulation can lead to a retardation of heat creation that would result in a locked rotor condition. Further, in accordance with the present disclosure, a larger powered accumulator of a lower ultimate pulse width modulation limit may be utilized to momentarily unstick a locked motor. The motor may reverse direction to result in backward motion of the firing driver and then may reverse direction again to restart forward motion of the firing driver to get through a portion of tissue where mechanical work needed. Alternatively, or additionally, the motor can reverse direction to result in backward motion of the firing driver and then reverse direction again to re-build up dynamic inertia of the firing driver.

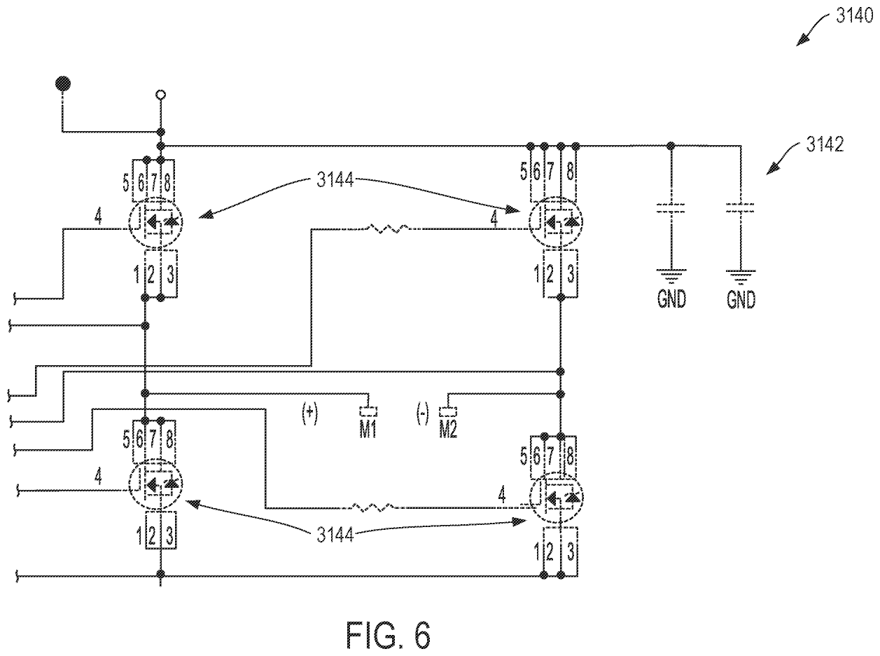

In accordance with the present disclosure, voltage drop can be minimized by accumulating power within the system. Additionally, in accordance with the present disclosure, accumulation of power within the system can be accomplished using a circuit 3140, illustrated in FIG. 6, that includes capacitors 3142 is series with field effect transistors 3144 (FETs). Utilizing the circuit 3140 with capacitors in series with FETs 3144, adds power to the system when the voltage potential of the power source begins to drop. The capacitors may provide a short term power boost, such as 1-5% of the total drive, for handling overload force conditions. Additionally, in accordance with the present disclosure, the capacitors may help during in-rush or small calcified areas of the tissue that cause spikes in the force required to fire the firing driver. It should be understood that the circuit 3140 is just one illustrative embodiment with capacitors in series with FETs 3144 and that various other circuits are contemplated by the present disclosure.

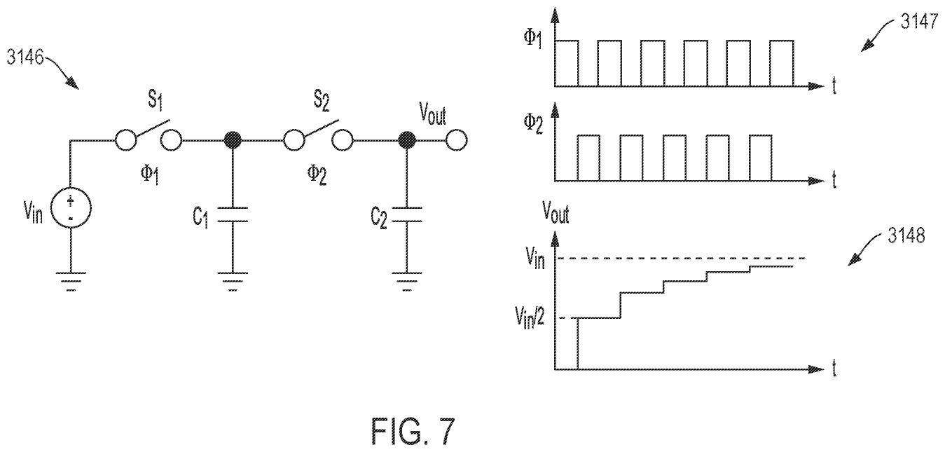

In accordance with the present disclosure, accumulation of power within the system may be accomplished by using a switching capacitor circuit 3146, illustrated in FIG. 7, which is a discrete-time circuit that exploits the charge transfer in and out of a capacitor as controlled by switches. The switching activity 3147 is controlled by non-overlapping clocks such that the charge transfer in and out is well defined and deterministic. The switching capacitor circuit 3146 provides a stepwise voltage output 3148 to the motor to provide an energy boost during the firing stroke.

In accordance with the present disclosure, accumulation of power within the system may be accomplished using capacitors plus pulse width modulation signals that are additive. A duty cycle of the motor may be selected such that the capacitors are re-charged after each drain, which reduces, or diminishes, power source voltage drop. Additionally, or alternatively, the duty cycle may be selected between 30-50%. A selected balance between pulse withdrawn activations and the capacitor sizing leads to a reduction, or elimination, or voltage sag from the power source.

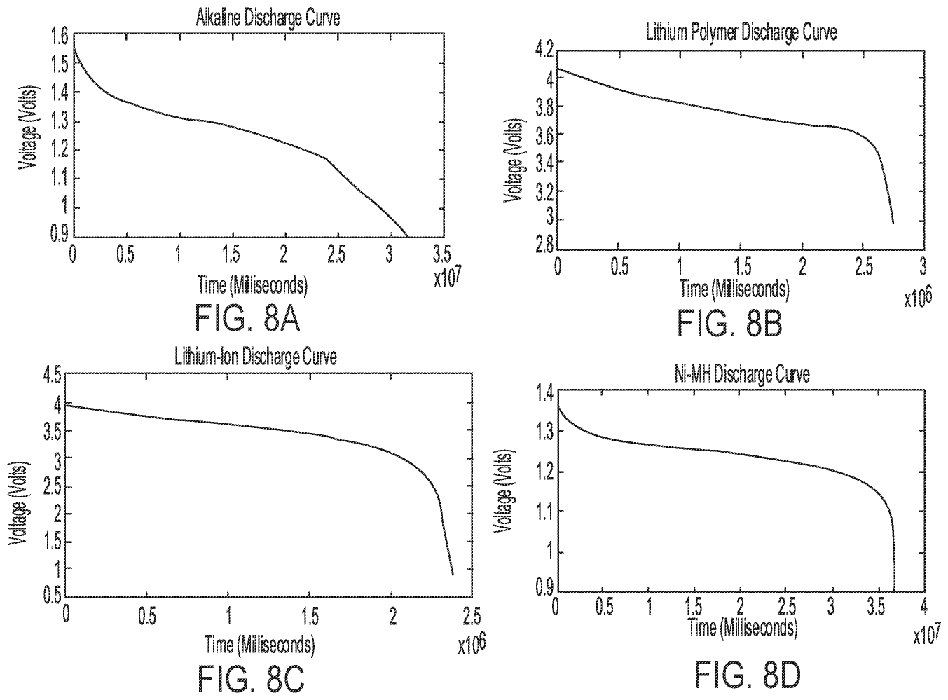

In accordance with the present disclosure, accumulation of power within the system may be based on the chemistry of the system. Additionally, or alternatively, accumulation of power within the system may be based on the duty cycle of the power source, the chemistry of the power source, or the power source chemistry recovery, or a combination thereof. For example, a material for a power source may be selected based on the observed power source voltage drop under load over time. FIGS. 8A-8D illustrate loaded power source voltage drop over time of several power sources utilizing different battery materials. FIG. 8A illustrates a discharge curve over time for a power source utilizing alkaline battery cells that includes a maximum voltage potential of ˜1.6V. FIG. 8B illustrates a discharge curve over time for a power source utilizing lithium polymer battery cells that includes a maximum voltage potential of ˜4.1V. FIG. 8C illustrates a discharge curve over time for a power source utilizing lithium-ion battery cells that includes a maximum voltage potential of ˜4V. FIG. 8D illustrates a discharge curve over time for a power source utilizing nickel-metal hydride (Ni-MH) battery cells that includes a maximum voltage potential of ˜1.35V. Based on the observed voltage drops over time, a user can select an appropriate power source for a desired surgical outcome.

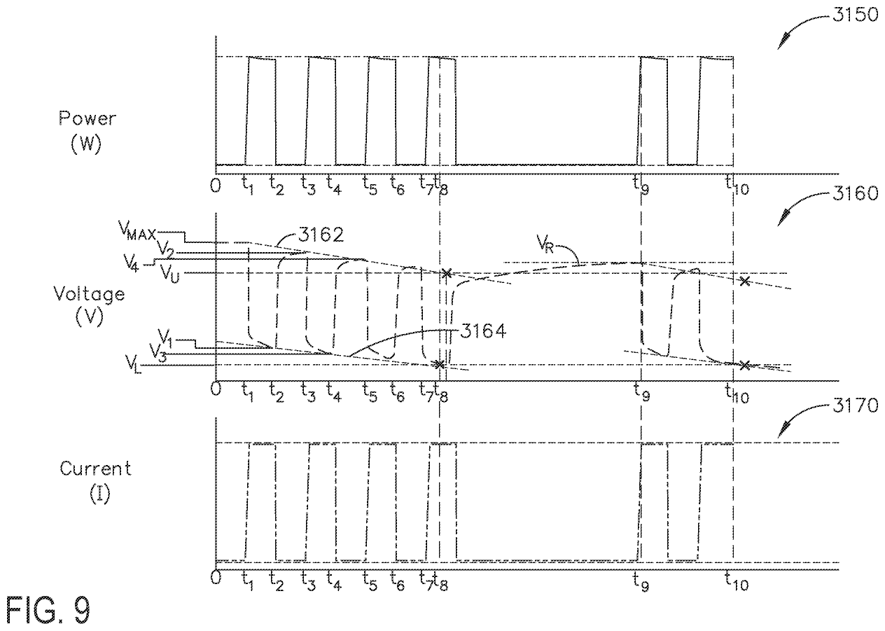

In accordance with the present disclosure, accumulation of power within the system may be based on motor power consumption, chemistry of the power source, or recovery of the power source, or a combination thereof. The control system can monitor the voltage potential of the power source over the course of a firing stroke in order to determine if the firing stroke should be paused to allow the power source to recover. Graphs 3150, 3160, 3170 shown in FIG. 9 show an exemplary firing stroke of a firing driver through a staple cartridge. Graph 3150 illustrates the power output from a power source over time. The power source may comprise a CR123A primary lithium battery. Graph 3160 illustrates the voltage potential of the power source over time. Graph 3170 illustrates the current output of the power source over time. As shown in graph 3160, the power source has a maximum voltage potential of V_{MAX}.

Based on the firing stroke power source, a control system comprising a controller 3033, 3051, sets a power source upper threshold V_{U}, a power source lower threshold V_{L}, and a power source recovery threshold V_{R}. In accordance with the present disclosure, the control system may interrogate the power source to determine a type of the power source. Based on the determination, the control system retrieves the power source upper threshold V_{U}, the power source lower threshold V_{L}, and the power source recovery threshold V_{R} from a memory. Alternatively, the power source upper threshold V_{U}, the power source lower threshold V_{L}, and the power source recovery threshold V_{R} are user defined. Alternatively, the power source upper threshold V_{U}, the power source lower threshold V_{L}, and the power source recovery threshold V_{R} are based on the maximum voltage potential of the power source. Alternatively, the power source upper threshold V_{U}, the power source lower threshold V_{L}, and the power source recovery threshold V_{R} are a predefined percentage of the maximum voltage potential of the power source. Alternatively, the power source upper threshold V_{U}, the power source lower threshold V_{L}, and the power source recovery threshold V_{R} are the same regardless of the power source used by the motor.

At time t₁, the control system initiates an adaptive firing algorithm based on a user providing an input to the control system. In accordance with the present disclosure, the adaptive firing algorithm may be similar to other adaptive firing algorithms discussed elsewhere herein. The adaptive firing algorithm may be stored in a memory 3053, and executable by a processor 3052. In accordance with the present disclosure, the user may provide the input to the control system via an input interface at display 3068. Based on the initiation of the adaptive firing algorithm, the adaptive firing algorithm applies a first duty cycle to the motor.

Based on the initiation of the adaptive firing algorithm, at t₁, the motor is transitioned to an on state for a first period according to the first duty cycle, causing the motor to draw power from the power source to drive a firing driver, such as firing driver 3024, toward its fired position. During the first period (from t₁ to t₂), the power source applies a maximum current I_{MAX} to the motor and the voltage potential of the power source drops from V_{Max} to a first lower voltage potential V₁. In accordance with the present disclosure, the control system may detect, or measure, the applied currents and voltages using a current sensor and a voltage sensor, respectively.

At time t₂, the control system detects, via the voltage sensor, that the voltage potential of the power source has dropped to a first dropped voltage potential V₁. In accordance with the present disclosure, the control system may store the first dropped voltage potential V₁ in the memory for subsequent evaluations. The control system compares the first lower voltage potential V₁ to the power source lower threshold V_{L} to determine if the first lower voltage potential V₁ is above or below the power source lower threshold V_{L}. Based on the control system determining that the first lower voltage potential V₁ is above the power source lower threshold V_{L}, the firing algorithm maintains the first duty cycle. Based on the control system determining that the first lower voltage potential V₁ has reached or dropped below the power source lower threshold V_{L}, the control system adjusts the firing algorithm. As shown in graph 3160, since the first dropped voltage potential V₁ is determined to be above the power source lower threshold V_{L}, the motor is transitioned to an “off” state for a second period according to the first duty cycle. During the second period (from t₂ to t₃), the voltage potential of the power source recovers to a first recovered voltage potential V₂.

At time t₃, the control system detects, via the voltage sensor, that the voltage potential of the power source has recovered to a first recovered voltage potential V₂. In accordance with the present disclosure, the control system may store the first recovered voltage potential V₂ in the memory for subsequent evaluations. The control system compares the first recovered voltage potential V₂ to the power source upper threshold V_{U} to determine if the first recovered voltage potential V₂ is above or below the power source upper threshold V_{U}. Based on the control system determining that the first recovered voltage potential V₂ is above the power source upper threshold V_{U}, the firing algorithm maintains the first duty cycle. Based on the control system determining that the first recovered voltage potential V₂ has reached or dropped below the power source upper threshold V_{U}, the control system adjusts the firing algorithm, as will be discussed in more detail below. As shown in graph 3160, since the first recovered voltage potential V₂ is determined to be above the power source upper threshold V_{U}, the motor is transitioned to the on state for a third period according to the first duty cycle. During the third period (from t₃ to t₄), the power source supplies the maximum current I_{MAX} to the motor and the voltage potential of the power source drops to a second dropped voltage potential V₃.

In accordance with the present disclosure, the control system may evaluate the two data points (e.g., the maximum voltage potential V_{Max} and the first recovered voltage potential V₂) and may project 3162 an anticipated recovered voltage potential over time utilizing the first duty cycle. Based on the projected voltage drop over time, the control system predicts a time that the recovered voltage potential of the power source is expected to fail to reach the power source upper threshold V_{U} prior to a later transition of the motor to an “on” state. In accordance with the present disclosure, the surgical system may comprise a display 3068, and the control system may display the predicted time on the display.

In accordance with the present disclosure, the control system may adjust the duty cycle of the firing algorithm based on the prediction. Additionally, based on the prediction, the control system may adjust the algorithm to change the duty cycle of the motor so as to maintain the recovered voltage potential above the power source recovery threshold V_{U} prior to subsequent transitions of the motor to the on state for the remainder of the firing stroke. In accordance with the present disclosure, based on the prediction, the control system may adjust the duty cycle to increase a length of the “off” pulses of the duty cycle. Additionally, or alternatively, based on the prediction, the control system may adjust the duty cycle to increase a length of the “on” pulses of the duty cycle. Further, in accordance with the present disclosure, based on the prediction, the control system may adjust the duty cycle to increase a length of the “on” pulses and the “off” pulses of the duty cycle.

At time t₄, the control system detects, via the voltage sensor, the drop in voltage potential of the power source to a second lower voltage potential V₃. The control system can store the second lower voltage potential V₃ in the memory for subsequent evaluations. The control system compares the second lower voltage potential V₃ to the power source lower threshold V_{L} to determine if the second lower voltage potential V₃ is above the power source lower threshold V_{L}. Based on the control system determining that second lower voltage potential V₃ is above the power source lower threshold V_{L}, the firing algorithm maintains the first duty cycle. Based on the control system determining that second lower voltage potential V₃ has reached or dropped below the power source lower threshold V_{L}, the control system adjusts the firing algorithm. Since the second lower voltage potential V₃ is determined to be above the power source lower threshold V_{L}, the motor is transitioned to an “off” state for a fourth period according to the first duty cycle as shown in graph 3160. During the fourth period (from t₄ to t₅), the voltage potential of the power source recovers to a second recovered voltage potential V₄.

In accordance with the present disclosure, the control system may evaluate the two data points, the first lower voltage potential V₁ and the second lower voltage potential V₃, and may project 3164 an anticipated voltage potential drop over time utilizing the first duty cycle. Based on the projected voltage drop over time, the control system predicts a time that the voltage potential of the power source is expected to drop below the power source lower threshold V_{L} during a subsequent “on” state of the motor. In accordance with the present disclosure, the control system may display the predicted time on the display. Additionally, or alternatively, the control system may display both predicted times (predicted time that power source will fail to recover to the power source upper threshold V_{U} and predicted time that power source will drop below the power source lower threshold V_{L}) to inform a user as to which event is expected to occur first.

In accordance with the present disclosure, the control system may adjust the duty cycle of the firing algorithm based on the prediction. Additionally, in accordance with the present disclosure, based on the prediction, the control system may adjust the algorithm to control the duty cycle of the motor to maintain the lower voltage potential above the power source lower threshold V_{L} for the remainder of the firing stroke. Further, in accordance with the present disclosure, based on the prediction, the control system may adjust the duty cycle to increase a length of the “off” pulses of the duty cycle. Additionally, in accordance with the present disclosure, based on the prediction, the control system may adjust the duty cycle to increase a length of the “on” pulses of the duty cycle. Further, in accordance with the present disclosure, based on the prediction, the control system may adjust the duty cycle to increase a length of the “on” pulses and the “off” pulses of the duty cycle.

As shown in graph 3160, from time t₄ to t₆, the control system maintains the first duty cycle to transition the motor to the “off” state (t₄ to t₅), the “on” state (t₅ to t₆), and the “off” state (t₆ and t₇). At each time point, the control system compares the detected voltage potential to a respective threshold (power source upper threshold V_{U} and power source lower threshold V_{L}) to determine if the first duty cycle should be maintained. At each time point, the control system stores the detected voltage potential in the memory and uses the data points to update the respective projections 3162, 3164 and predictions.

As shown in graph 3160, at time t₇, the motor is transitioned to the “on” state for a period according to the first duty cycle, which causes the voltage potential to drop to a third lower voltage potential. During the “on” state, as also seen in graph 3160, the voltage potential of the power source drops below the power source lower threshold V_{L}. In accordance with the present disclosure, despite dropping below the power source lower threshold V_{L}, the firing algorithm can maintain the motor in the on state according to the first duty cycle to allow the motor to finish the “on” pulse. Further, in accordance with the present disclosure, upon detecting the voltage potential dropping below the power source lower threshold V_{L}, the control system can transition the motor to the “off” state, cutting short the “on” pulse, to allow the power source to recover for a recovery period.

In the firing algorithm, the control system compares the third lower voltage potential to the power source lower threshold V_{L} to determine if the third lower voltage potential is above or below the power source lower threshold V_{L}. Since the third lower voltage potential is determined by the control circuit to have dropped below the power source lower threshold V_{L}, the control system transitions the motor to the “off” state and maintains the motor in the “off” state for a recovery period. In accordance with the present disclosure, the recovery period can correspond to the time required for the voltage potential of the power source to recover from the third lower voltage potential to the power source recovery threshold V_{R}. Further, in accordance with the present disclosure, the recovery period may be longer than the “off” pulse for the first duty cycle. As shown in graph 3160, at time t₉, based on the control system detecting that the voltage potential of the power source has reached the power source recovery threshold V_{R}, the control system transitions the motor to the on state and resumes applying the firing algorithm with the first duty cycle. In accordance with the present disclosure, based on the control system detecting that the voltage potential of the power source has reached the power source recovery threshold V_{R}, the control system may transition the motor to the on state and may apply a second duty cycle that is different than the first duty cycle.

While FIG. 9 illustrates a scenario where the voltage potential drops below the power source lower threshold V_{L} during an on-pulse, it should be understood that the control system operates in a similar manner when the voltage potential fails to reach the power source upper threshold V_{U} during an off-pulse. In accordance with the present disclosure, based on the voltage potential failing to reach the power source upper threshold V_{U} during the off-pulse, the control system may maintain the motor in the “off” state for a recovery period to allow the voltage potential of the power source to reach the power source recovery threshold V_{R} prior to re-implementing the first duty cycle. Based on the control system detecting that the voltage potential of the power source has reached the power source recovery threshold V_{R}, the control system transitions the motor to the “on” state and resumes applying the firing algorithm with the first duty cycle. In accordance with the present disclosure, based on the control system detecting that the voltage potential of the power source has reached the power source recovery threshold V_{R}, the control system may transition the motor to the “on” state and applies a second duty cycle that is different than the first duty cycle.

After re-implementing the first duty cycle, the control system continues to compare the detected voltage potentials to the respective thresholds at each on and off pulse to determine if the first duty cycle should be maintained for the remainder of the firing stroke. As shown in graph 3160, at time t₁₀, the firing stroke of the firing driver ends before either thresholds are reached or dropped below, and therefore, no additional recovery periods are needed. However, it should be understood that, had a respective threshold been reached or dropped below, the control system would operate in a similar manner as described above, in which the motor is maintained in an off state for a recovery period to allow the voltage potential of the power source to recover to the power source recovery threshold V_{R}.

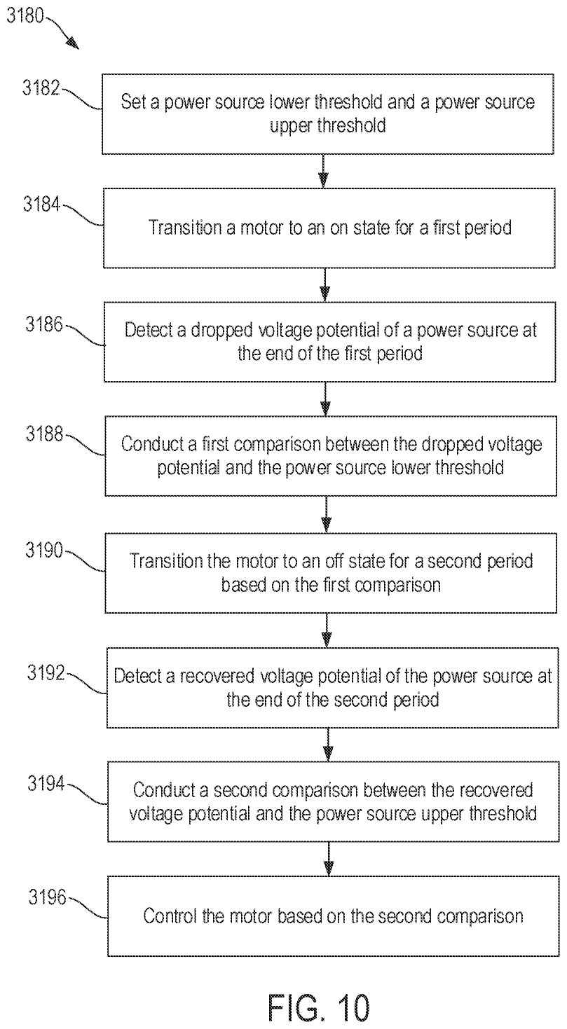

FIG. 10 shows a method 3180 for controlling a surgical system, according to the present disclosure. The method 3180 comprises setting 3182 a power source lower threshold and a power source upper threshold. In accordance with the present disclosure, a control system comprising a controller 3033, 3051, can set a power source lower threshold V_{L} and a power source upper threshold V_{U}. The method 3180 may include setting a power source recovery threshold, such as power source recovery threshold V_{R}. Further, in accordance with the present disclosure, the control system may set the thresholds by retrieving the thresholds from a memory.

The method 3180 comprises transitioning 3184 a motor to an “on” state for a first period. In accordance with the present disclosure, the control system may implement a first duty cycle which turns the motor on for a first period according to the duty cycle. The motor may comprise a firing motor 3056 that drives a firing driver 3024 toward a fired position to deploy staples 3023 from a staple cartridge 3008 when the motor is in an “on” state.

The method 3180 comprises detecting 3186 a dropped voltage potential of a power source at the end of the first period. The control system can interrogate a voltage sensor 3039 to determine the voltage potential of a power source 3041, 3055 that powers the motor. During the “on” state of the motor, the voltage potential of the power source drops from a maximum, or recovered, voltage potential to a lower voltage potential.

The method 3180 comprises conducting 3188 a first comparison between the lower “dropped” voltage potential and the power source lower threshold. In accordance with the present disclosure, the control system may compare the lower “dropped” voltage potential to the power source lower threshold to determine if the lower “dropped” voltage potential is above or below the power source lower threshold.

The method 3180 comprises transitioning 3190 the motor to an “off” state for a second period based on the first comparison. In accordance with the present disclosure, based on the lower “dropped” voltage potential being above the power source lower threshold, the control system may maintain the current duty cycle of the motor and allows the motor to remain “off” for a period according to the duty cycle. Additionally, in accordance with the present disclosure, based on the dropped voltage potential reaching or falling below the power source lower threshold, the control system may transition the motor to the “off” state for a recovery period, which may be longer than the current duty cycle time. The recovery period may correspond to the time required for the voltage potential of the power source to recover to the power source recovery threshold.

The method 3180 comprises detecting 3192 a recovered voltage potential of the power source at the end of the second period. In accordance with the present disclosure, once the second period has elapsed, the control system may interrogate the voltage sensor to determine the voltage potential of the power source. As discussed elsewhere herein, during the “off” state of the motor, the voltage potential of the motor rises, or recovers, from a lower “dropped” voltage potential.

The method 3180 comprises conducting 3194 a second comparison between the recovered voltage potential and the power source upper threshold. The control system can compare the recovered voltage potential to the power source upper threshold to determine if the lower “dropped” voltage potential has reached, or is below, the power source upper threshold.

The method 3180 comprises controlling 3196 the motor based on the second comparison. In accordance with the present disclosure, based on the recovered voltage potential reaching the power source upper threshold, the control system may allow the motor to maintain the current duty cycle of the motor, and thus, may turn the motor back to the “on” state after the second period has elapsed. Alternatively, based on the recovered voltage potential failing to reach the power source upper threshold, the control system may maintain the motor in the “off” state to allow for additional recovery of the motor. Additionally, in accordance with the present disclosure, the control system may maintain the motor in the “off” state after the second period for a recovery period, which may correspond to the time required for the voltage potential of the power source to recover to the power source recovery threshold.

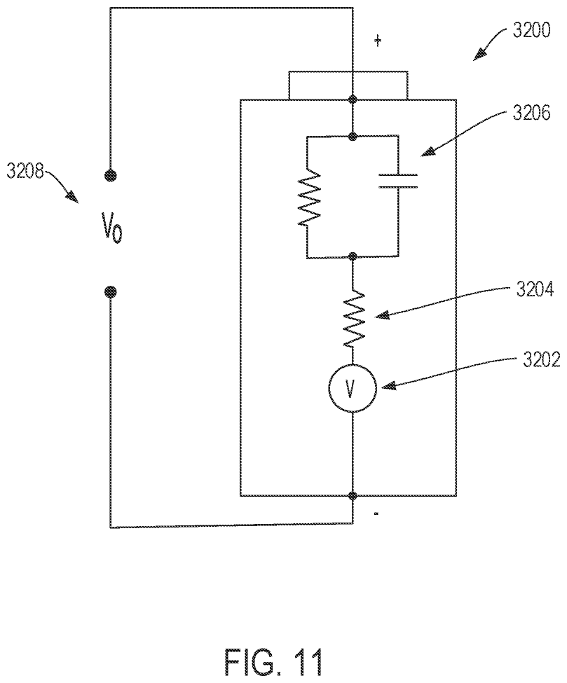

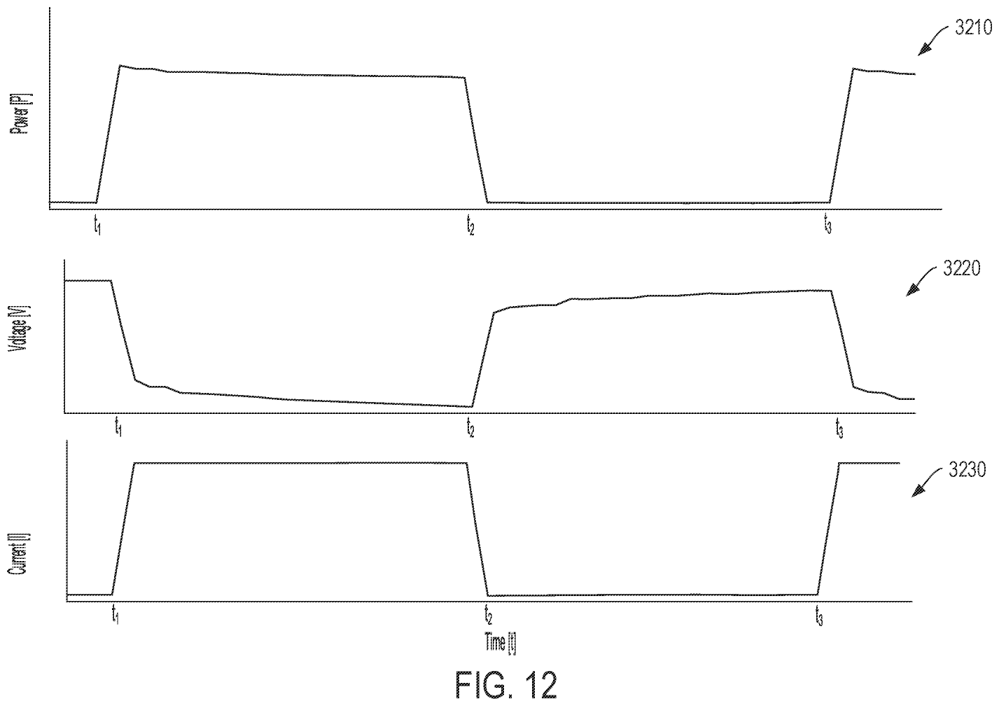

FIG. 11 shows a battery 3200, according to the present disclose. The battery 3200 comprises a circuit including a voltage source 3202 with a voltage potential of V_{Max}, a resistor 3204, a resistor-capacitor (RC) circuit 3206 in series with the voltage source 3202 and the resistor 3204, and a voltage output V₀ 3208 in series with the battery 3200. FIG. 11 illustrates graphs 3210, 3220, 3230 of an exemplary firing stroke of a firing driver where the battery 3200 provides power to a firing motor that drives the firing driver.

At time t₁, a control system comprising a controller 3033, 3051 applies a firing algorithm, which causes the firing motor to transition to an “on” state and draw current from the battery 3200 to drive the firing driver toward the distal position. From time t₁ to t₂, as shown in graphs 3210, 3220, 3230, the voltage output 3208 of the battery 3200 drops from Vₘₐₓ to V₁, the current drawn by the motor increases and is maintained at I_{Max}, and the power consumed by the motor increases to P_{Max} and drops to P₁. At time t₂, the firing algorithm transitions the motor to the “off” state, causing the motor to stop drawing current from the battery 3200. From time t₂ to t₃, based on the chemistry and the circuitry of the battery 3200, the voltage potential of the battery 3200 first sharply rises and then steadily rises to a first recovered voltage V_{R}.

In accordance with the present disclosure, the period from t₂ to t₃ may be a variable period. Additionally, or alternatively, the period from t₂ to t₃ may be a predetermined period. Additionally, or alternatively, the period may be based on an applied duty cycle to the motor. Additionally, or alternatively, the period from t₂ to t₃ may be based on a magnitude of the voltage potential drop from t₁ to t₂. Additionally, or alternatively, the period from t₂ to t₃ may be based on a rate at which the voltage potential dropped from t₁ to t₂. Additionally, or alternatively, the period from t₂ to t₃ may be based on the time required by the battery 3200 to recover a threshold amount of voltage potential from V₁. The time from t₂ to t₃ may be based on a rate at which the voltage potential recovers from V1.

At time t₃, the firing algorithm transitions the firing motor to the “on” state again such that the motor draws current from the battery 3200 to resume driving the firing driver toward the fired position.

Voltage drop is minimized by accumulating power within the system. In accordance with the present disclosures, accumulation of power within the system may be accomplished by storing energy during off states of the power source. An “off” state may comprise an “off” pulse of a pulse width modulation signal. Further, additionally, or alternatively, an “off” state may comprise a state in between firing strokes of a firing driver between the conclusion of a first firing stroke and the start of a second firing stroke. In accordance with the present disclosure, energy may be stored in the system using capacitors. Energy stored within a capacitor is given by the classic equation U=0.5(C)(V²), where U is the capacitor energy, C is the capacitance of the capacitor, and V is the voltage. Based on this equation, the same capacitor charged to a higher voltage will store exponentially more energy within a system.

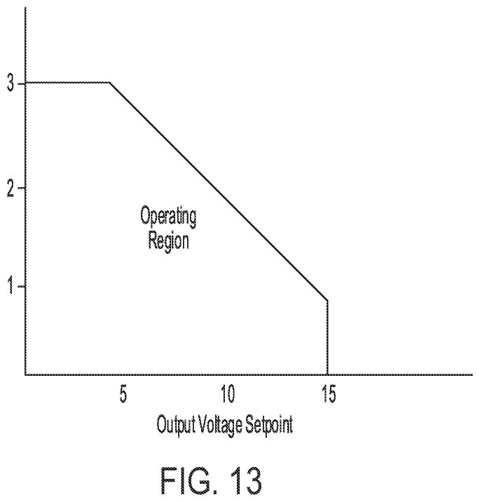

The control system can comprise a voltage convertor to control voltage that is applied to the motor from the power source, which reduces power source voltage drop. A constraint of many voltage converter methodologies is a limitation of load curves. Total output power is fixed for variable voltages, which impacts the current available for sourcing. FIG. 13 illustrates a load curve profile for a 15 W power source. Utilizing a voltage converter, a direct bearing on what the system can deliver is known depending on a set the input voltage.

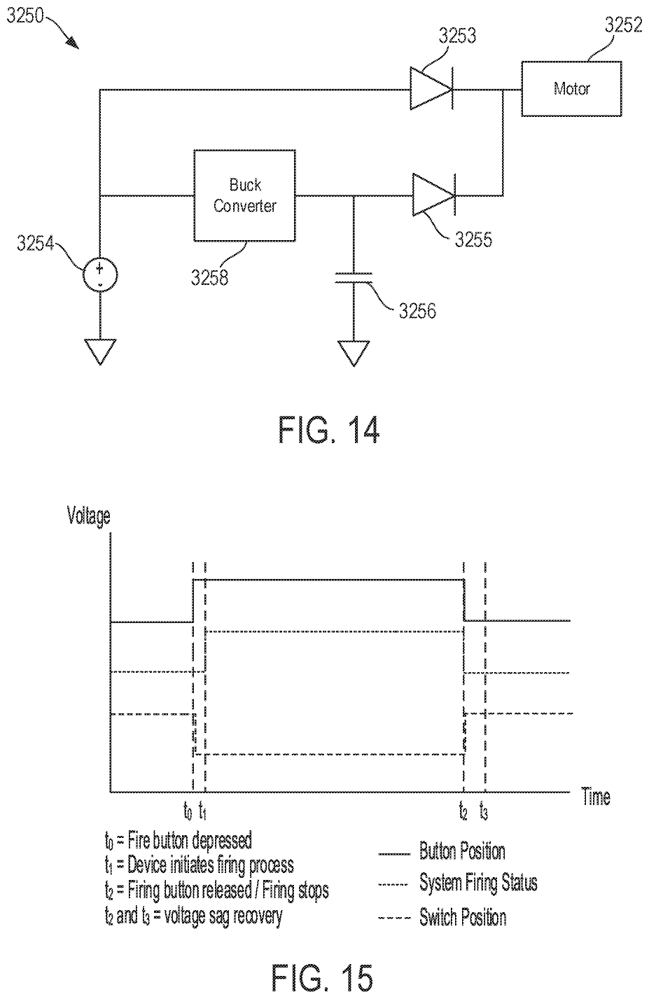

In accordance with the present disclosure, power can be accumulated in the system using a storage capacitor. The storage capacitor may be selectively connected to the motor using active or passive components (e.g., via Diode OR'ing) to allow for very low impact on inrush capabilities. Further, in accordance with the present disclosure, referring to FIG. 14, a circuit 3250 that includes a motor 3252, a power source 3254 that provides power to the motor 3252, and a diode 3253 between the power source 3254 and the motor 3252. The circuit 3250 includes a storage capacitor 3256 in electrical communication with the motor 3252, a buck converter 3258 that steps down voltage from the power source 3254 to the storage capacitor 3256, and a diode 3255 between the storage capacitor 3256 and the motor 3252.

In operation, during an “off” state of the motor 3252, current flows from the power source 3254 to the storage capacitor 3256 via the buck converter 3258 to charge the storage capacitor 3256. When the motor 3252 transitions to an “on” state to drive a firing driver, current flows from the storage capacitor 3256 and the power source 3254 to the motor 3252 to mitigate motor 3252 inrush current. After the initial transition to the “on” state, the current flows from the power source 3254 to the motor 3252 to drive the firing driver. When the motor 3252 is again transitioned to the “off” state, the current once again flows from the power source 3254 to the storage capacitor 3256 via the buck converter 3258 to charge the storage capacitor 3256.

In accordance with the present disclosure, the storage capacitor 3256 may have a much lower internal resistance when compared to the power source 3254. Accordingly, the voltage drop associated with the storage capacitor 3256 will be much smaller than the power source 3254 voltage drop. The storage capacitor 3256 will aid in keeping the voltage potential to the motor 3252 high, which provides the motor 3252 with more power to complete the firing stroke.

In accordance with the present disclosure, a variable output DC/DC converter can be placed in series with a switching circuit, such as conventional mechanical switch, or electronic switch such as a MOSFET, as examples. Based on the motor being in an “off” state, the DC/DC converter will change its output to a higher voltage to charge capacitors and store energy. A diode OR will drop the voltage to the appropriate level.

Referring now to FIG. 15, a graph 3260 is provided that illustrates button position of a surgical system, a switch position, and firing status of a motor over time. At time t₀, a user actuates a button of the surgical system at an input interface at display 3068. Based on the actuation, the switch transitions from an open state to a closed state to electrically couple a storage capacitor to the motor, which causes the capacitor to apply a voltage to the motor. At time t₀, an international delay is introduced, using the aforementioned switching circuit, which provides a slight delay before the motor draws current from the power to supply power to the motor and begin driving the firing driver. In accordance with the present disclosure, the slight delay may provide inrush current mitigation. After the intentional delay, at time t₁, the motor transitions to an “on” state to drive the motor. At time t₂, the power source ceases providing power to the motor to transition the motor to the “off” state, the switch transitions back to the “off” state, and the button returns to an unactuated state. Based on the changes, the power source recovers voltage potential and the capacitor is refilled in preparation for a subsequent firing cycle.

In accordance with the present disclosure, accumulation of power within the system can be accomplished by managing power accumulation available within the power source. For instance, due to motor inrush current, the current drawn by the motor from the power source at the beginning of any firing stroke will be substantial. This places a significant limitation on the energy storage capabilities for subsequent firings of the motor. Utilizing a starting consistent operational parameter allows the power source to “recover” and accumulate energy in between firing strokes to provide a more consistent firing outcome over multiple firing strokes of the firing driver. Accordingly, it is desirable that a user of the system be aware of the power accumulation of the power source prior to initiating a subsequent firing stroke in order to understand that the device may perform less than normal unless a sufficient amount of power has been recovered/re-accumulated.

In accordance with the present disclosure, a control system comprising controller 3033, 3051, can monitor an amount of energy accumulated in a power source 3055 after the conclusion of the firing strokes of a firing driver 3024. For instance, a user may provide a first firing input to the control system at an input interface of display 3068. Based on receiving the first firing input, the control system controls a firing motor 3056 to drive the firing driver through a first firing stroke from the proximal, unfired position to the distal, fired position.

Based on the conclusion of the first firing stroke, the control system interrogates a sensor to monitor an amount of power that is re-accumulating within the power source over time. In accordance with the present disclosure, the sensor may comprise a voltage sensor 3054 and the control system may monitor the voltage potential of the power source over time using the voltage sensor. The control system displays the monitored amount of power on a display 3068 to notify the user of the amount of power within the power source. In accordance with the present disclosure, the control system may display the maximum power of the power source on the display. Based on visual cues, a user can determine whether to proceed with a second firing stroke of the firing driver, or wait an additional period prior to the initiation of the second firing stroke to allow for additional power accumulation in the power source.

In accordance with the present disclosure, the control system may set a recovery threshold of the power source. The recovery threshold may correspond to a threshold amount of energy required of the power source to accomplish a subsequent firing stroke. Alternatively, the recovery threshold may correspond to a threshold amount of energy required to allow the firing driver to operate in the same manner as in the first firing stroke. Alternatively, the recovery threshold may be stored in a memory and retrievable by the control system. Alternatively, the recovery threshold may be user defined. Alternatively, the recovery threshold may be a predefined percentage of the maximum voltage potential of the power source. Alternatively, the recovery threshold may comprise a recovery voltage potential of the power source.

In accordance with the present disclosure, the control system may display the recovery threshold on the display to enable the user to visually compare the monitored amount of power of the power source to the recovery threshold. Based on the visual comparison, a user can choose to wait until the power has reached the recovery threshold until actuating a second firing stroke of the firing driver. The user also can choose to actuate the second firing stroke of the firing driver prior to the monitored power reaching the recovery threshold; however, the displayed power will indicate to the user that the system will perform less than normal as the power source was not allowed to re-accumulate to the recovery threshold.

In accordance with the present disclosure, the control system may include a firing lockout to prevent the user from performing another firing stroke until a sufficient amount of energy has been re-accumulated in the power source. Further, in accordance with the present disclosure, the control system may compare the monitored power to the recovery threshold based on a user providing a second firing input to the input interface. Based on the monitored power being less than the recovery threshold, the control system ignores the second firing input and prevents the motor from driving the firing driver through the firing stroke because a sufficient amount of energy has not been re-accumulated in the power source. The control system issues a notification on a display 3068 indicating that a sufficient amount of energy has not been re-accumulated and that additional time is required until the subsequent firing stroke can be initiated. Based on the monitored power reaching or exceeding the recovery threshold, the control system allows the firing stroke to commence and controls the motor to drive the firing driver through the subsequent firing stroke. In accordance with the present disclosure, the control system may display a notification on the display to inform the user that the firing system is available for actuation based on the monitored energy reaching the recovery threshold.

In accordance with the present disclosure, the control system may include a firing lockout to prevent the user from performing another firing stroke until a threshold period has elapsed from the conclusion of a previous firing stroke. The threshold period may be fixed. Further, in accordance with the present disclosure, the power source may comprise an RC circuit and the RC time constant dictates the threshold period. Based on the RC time constant, the control system prevents a user from initiating a subsequent firing stroke for a fixed period to guarantee that the capacitor has had sufficient time to recharge over the period equal to the RC time constant. The threshold period may be variable. Additionally, in accordance with the present disclosure, based on system characterization, and known RC time constant recharge rates, the firing lockout may lock out the user from actuating the firing system for a variable period based on a calculation of predicted energy storage in the power source. Based on the threshold period elapsing, the control system displays a notification on the display to inform the user that the firing system is available for actuation and allows the user to initiate the subsequent firing stroke.

The control system can monitor time, intensity, and/or duration of power draw from the power source during use thereof. Based on the monitored parameters, the control system controls when, and how much, subsequent power draws are allowed or enabled. Such control allows for electrolyte recovery in the power source, as well as allows for heat dissipation of the cells of the power source, which improves the output capacity for the next power usage of the power source, such as during a subsequent firing stroke of the firing driver.

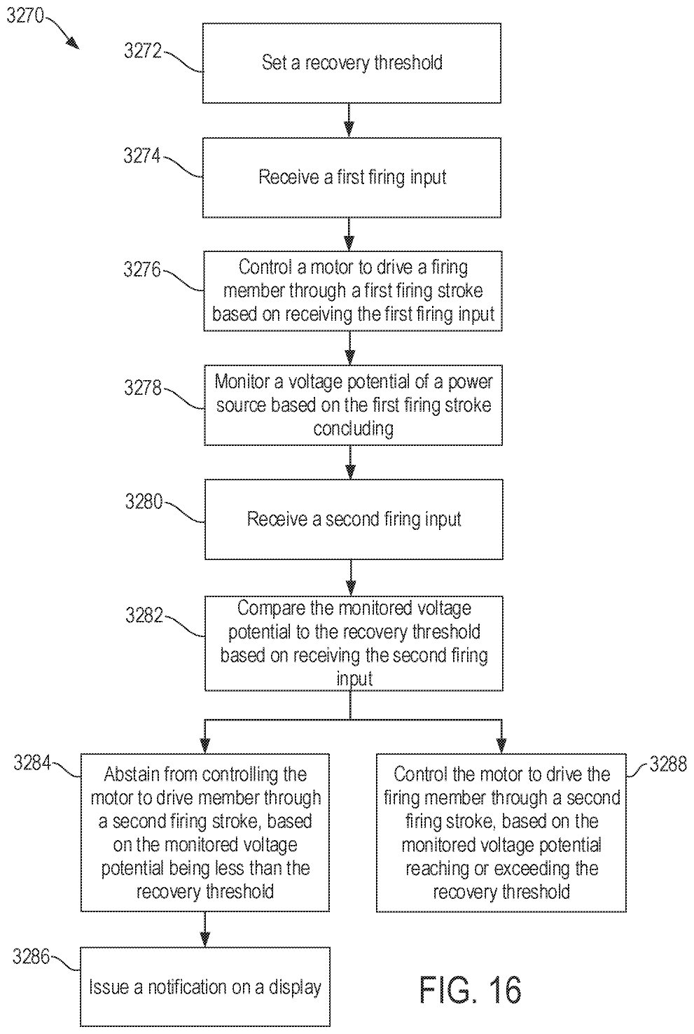

FIG. 16 illustrates a method 3270 for controlling a surgical system, according to the present disclosure. The method 3270 comprises setting 3272 a recovery threshold. In accordance with the present disclosure, a control system comprising a controller 3033, controller 3051 can set a recovery threshold, as described elsewhere herein, such as by retrieving the recovery threshold from a memory. The recovery threshold may comprise a voltage potential threshold associated with a power source 3055.

The method 3270 comprises receiving 3274 a first firing input. A user provides a first firing input to a control system comprising a controller 3033, 3051 at an input interface of display 3068 in order to perform a first firing stroke of the firing driver.

The method 3270 comprises controlling 3276 a motor to drive a firing driver through a first firing stroke based on receiving the first firing input. Based on receiving the first firing input, the control system can control a firing motor 3056 to drive a firing driver 3024 through a first firing stroke, from the proximal, unfired position to the distal, fired position.

The method 3270 comprises monitoring 3278 a power source voltage potential at the end of conclusion the first firing stroke. The motor draws current from a power source 3055 to drive the firing driver through the firing stroke and a voltage sensor 3039 senses the voltage potential of the power source over time. The control system interrogates the voltage sensor after the firing stroke has concluded to determine the dropped voltage potential of the power source and to monitor the voltage recovery of the power source over time. In accordance with the present disclosure, the method may comprise displaying the monitored voltage potential on a display 3068 to enable a user to visually track the voltage recovery of the power source over time.

The method 3270 comprises receiving 3280 a second firing input. In accordance with the present disclosure, a user may provide the second firing input to a control system comprising a controller 3033, 3051 at the input interface in order to perform a second firing stroke of the firing driver.

The method 3270 comprises comparing 3282 the monitored voltage potential to the recovery threshold based on receiving the second firing input. In accordance with the present disclosure, based on the user attempting to initiate a second firing stroke of the firing driver by providing a second firing input to the input interface, the control system may compare the monitored voltage potential to the recovery threshold in order to determine if the firing driver should be allowed to advance through a second firing stroke.

The method 3270 comprises abstaining 3284 from controlling the motor to drive the firing driver through a second firing stroke based on the monitored voltage potential being less than the recovery threshold. In accordance with the present disclosure, based on the control system determining that the monitored voltage has not yet reached the recovery threshold, the control system may ignore the second firing input and may abstain from advancing the firing driver through the second firing stroke with the motor. The method 3270 comprises issuing 3286 a notification on a display. In accordance with the present disclosure, the control system may transmit a signal to the display to display a notification that informs the user that the power source has not yet reached the recovery threshold, and therefore, the second firing stroke cannot be completed at this time.

The method 3270 comprises controlling 3288 the motor to drive the firing driver through a second firing stroke based on the monitored voltage potential reaching or exceeding the recovery threshold. In accordance with the present disclosure, based on the monitored voltage potential reaching or exceeding the recovery threshold, the control system may control the motor to drive the firing driver through the second firing stroke. The method may comprise issuing a notification on the display based on the monitored voltage potential reaching the recovery threshold. Accordingly, a user is notified that the power source has recovered a sufficient amount of energy and that a second firing stroke can now be completed.

In accordance with the present disclosure, the method 3270 optionally may further comprise monitoring an elapsed period based on the first firing stroke concluding, comparing the elapsed period to a recovery time period based on receiving the second firing input, and performing an action, such as abstaining from driving the motor or controlling the motor to drive the firing driver through a second firing stroke, based on the comparison.

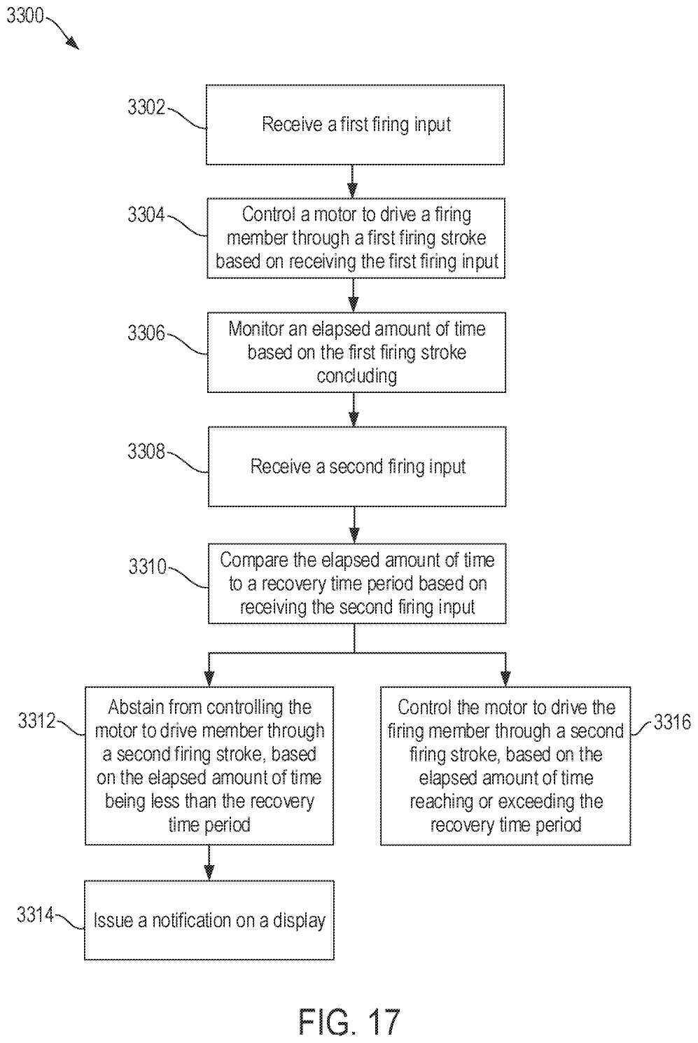

FIG. 17 shows a method 3300 for controlling a surgical system, according to the present disclosure. The method 3300 comprises receiving 3302 a first firing input. A user provides the first firing input to a control system comprising a controller 3033, 3051 at an input interface of display 3068 to initiate a first firing stroke of the firing driver.

The method 3300 comprises controlling 3304 a motor to drive a firing driver through a first firing stroke based receiving the first firing input. Based on receiving the first firing input, the control system can control a the firing motor 3056 to drive a firing driver 3024 through a first firing stroke from the proximal, unfired position to the distal, fired position.

The method 3300 comprises monitoring 3306 an elapsed period based on the first firing stroke concluding. In accordance with the present disclosure, based on the firing driver reaching the distal, fired position, the control system may initiate a timer, such as timer 3069, in order to measure an elapsed period from the conclusion of the firing stroke. As described elsewhere herein, after the conclusion of the firing stroke, a power source, such as power source 3055, that powers the motor begins to re-accumulate energy in preparation for a subsequent firing stroke.

The method 3300 comprises receiving 3308 a second firing input. In accordance with the present disclosure, a user can provide the second firing input to a control system, such as controller 3033 or controller 3051, at the input interface in order to perform a second firing stroke of the firing driver.

The method 3300 comprises comparing 3310 the elapsed period to a recovery time period based on receiving the second firing input. In accordance with the present disclosure, based on the user attempting to initiate a second firing stroke of the firing driver by providing the second firing input to the input interface, the control system may compare the elapsed period to the recovery time period in order to determine if the firing driver should be allowed to advance through a second firing stroke. By comparing the elapsed period to the recovery threshold, the control system determines if a sufficient amount of energy has been re-accumulated by the power source in order to complete the second firing stroke. In accordance with the present disclosure, the recovery period may be a predetermined period stored in a memory. Alternatively, or additionally, the recovery period may be a predetermined period input by a user at the input interface. Alternatively, or additionally, the recovery period may be based on the RC time constant of the power source. Alternatively, or additionally, the recovery period may be a variable period. In accordance with the present disclosure, based on the known RC time constant recharge rate of the power source, the control system can predict the time required before a subsequent firing stroke can be completed and the recovery period comprises this predicted period. Further, in accordance with the present disclosure, the recovery period may be a variable period based on the recharge rate of the power source.

The method 3300 comprises abstaining 3312 from controlling the motor to drive the firing driver through a second firing stroke based on the elapsed period being less than the recovery time period. Based on the control system determining that the elapsed period has not yet reached the recovery time period, the control system can ignore the second firing input and abstains from advancing the firing driver through the second firing stroke with the motor. The method 3300 comprises issuing 3314 a notification on a display. In accordance with the present disclosure, the control system may transmit a signal to the display to display a notification informing the user that the recovery time period has not yet elapsed and therefore, the second firing stroke cannot be completed at this time. The control system may display a countdown to inform a user as to how long until the subsequent firing stroke can be performed.

The method 3300 comprises controlling 3316 the motor to drive the firing driver through a second firing stroke based on the elapsed time reaching or exceeding the recovery time period. In accordance with the present disclosure, based on the elapsed period reaching or exceeding the recovery time period, the control system can control the motor to drive the firing driver through the second firing stroke. Further, in accordance with the present disclosure, the method may comprise issuing a notification on the display based on the elapsed period reaching the recovery time period. Accordingly, a user is notified that the power source has recovered a sufficient amount and that a second firing stroke can now be completed.

In accordance with the present disclosure, the method 3300 optionally may further comprise monitoring a power source voltage potential at the end of conclusion the first firing stroke, comparing the monitored voltage potential to the recovery threshold based on receiving the second firing input, and performing an action, such as abstaining from driving the motor or controlling the motor to drive the firing driver through a second firing stroke, based on the comparison.

In accordance with the present disclosure, voltage drop may be minimized by adjusting the configuration of the battery cells in the power source. Adjusting the configuration of the battery cells may comprise adjusting the number and/or configurations of the cells within the battery to minimize voltage drop.

Balancing the cells of the battery affects the impact of the battery output performance. The number of streams affects the impact of the battery output performance. In accordance with the present disclosure, voltage drop can be minimized by selectively placing some, or all, cells in parallel with one another. Alternatively, voltage drop can be minimized by selectively placing some, or all, cells in series with one another. Alternatively, voltage drop can be minimized by selectively placing some cells in series and some cells in parallel with one another. Paralleling cells increases the capacity of the output of the power source. Placing the cells in series increases the voltage of the output since series cells have internal resistance that are additive. Accordingly, a user can selectively place the battery cells in series and/or parallel based on the desired outcome. Alternatively, the combination of the battery cell chemistries may enhance the overall performance of the battery. For instance a secondary cell may be utilized in combination with a primary cell, where the secondary cell is used to handle the circuit inrush current. In accordance with the present disclosure, the control circuit can selectively switch between the primary cell and the secondary cell based on which of the cells is better suited for a desired need.

In accordance with the present disclosure, the control system may actively control the number of cells used during a firing stroke and makes adjustments “on the fly”. The control system may set a voltage drop threshold and may monitor the battery voltage drop during the firing stroke. Based on the voltage potential reaching or dropping below the voltage drop threshold, the control system changes a state of the battery to actively switch additional cells into the circuit to add additional power. In accordance with the present disclosure, the control system may utilize a first number of cells during an initial portion of a firing stroke. Based on the voltage potential reaching or dropping below the voltage drop threshold, the control circuit changes the state of the power source such that a second number of cells which is more than the first number of cells are utilized. The control system may complete this transition using switching circuitry to place additional cells in series and/or parallel with the first number of cells depending on a desired need. Additionally, in accordance with the present disclosure, based on the rate of change of the voltage drop, the control system may determine whether or not to switch from a primary power source configured to provide a first voltage potential to a second power source configured to provide a second voltage potential different than the first voltage potential.

In accordance with the present disclosure, the surgical system can include an auxiliary battery pack and the control system switches to the auxiliary battery pack if the primary power source voltage potential drops below a threshold level. Additionally, in accordance with the present disclosure, the control system can couple the auxiliary battery pack to the primary battery pack to enhance the power output of the system to a higher level than was previously available. For instance, the control system may interrogate a force sensor to determine if more power is necessary to complete a firing stroke. Based on the determination, the control circuit couples the auxiliary battery pack to the primary battery pack to provide the necessary power to complete the firing stroke.

In accordance with the present disclosure, the power source may comprise a battery that includes cells comprised of lithium ion. Alternatively, the cells may comprise lithium cobalt cells (NCA). Alternatively, the cells may comprise lithium nickel manganese cobalt cells (NMC). Alternatively, the cells may comprise lithium nickel cobalt aluminum cells (NCA). Alternatively, the cells may comprise lithium iron phosphate cells (LFP). Alternatively, the cells may comprise lithium manganese spinel cells (LMO). Alternatively, the cells may comprise lithium titanate cells (LTO). Alternatively, the cells may comprise lithium cobalt oxide cells (LCO).

Many of the surgical instrument systems described herein are motivated by an electric motor; however, the surgical instrument systems described herein can be motivated in any suitable manner. In accordance with the present disclosure, the surgical instrument systems described herein can be motivated by a manually-operated trigger, for example. In accordance with the present disclosure, the motors disclosed herein may comprise a portion or portions of a robotically controlled system. Moreover, any of the end effectors and/or tool assemblies disclosed herein can be utilized with a robotic surgical instrument system. U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535, for example, discloses several examples of a robotic surgical instrument system in greater detail.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one or more other embodiments without limitation. Also, where materials are disclosed for certain components, other materials may be used. According to various embodiments, a single component may be replaced by multiple components, and multiple components may be replaced by a single component, to perform a given function or functions. The foregoing description and following claims are intended to cover all such modification and variations.

Various aspects of the subject matter described herein are set out in the following numbered examples:

Example 1—A surgical system (3030, 3050) comprising an end effector (3002, 3038), a motor (3056), a power source (3041) configured to power the motor, a voltage sensor (3039) configured to sense the voltage potential of the power source, and a control circuit (3033, 3051) in operable communication with the motor and the voltage sensor. The end effector comprises a first jaw (3004, 3042) and a second jaw (3006, 3043) rotatable relative to the first jaw from an open position toward a closed position to capture tissue therebetween. The motor is operable between an on state in which the motor drives a motion at the end effector and an off state in which the motor ceases to the motion at the end effector. The control circuit is configured to set a power source lower threshold, transition the motor to the on state for a first period, detect a dropped voltage potential of the power source at the end of the first period, conduct a first comparison between the dropped voltage potential and the power source lower threshold, and transition the motor to the off state for a second period based on the first comparison.

Example 2—The surgical system of Example 1, wherein the control circuit is configured to set a power source recovery threshold, wherein the second period comprises a recovery period based on the dropped voltage potential reaching or dropping below the power source lower threshold, and wherein the recovery period corresponds to a time required for the voltage potential of the power source to recover to the power source recovery threshold.

Example 3—The surgical system of Examples 1 or 2, wherein the second period is identical to the first period based on the dropped voltage potential being greater than the power source lower threshold.

Example 4—The surgical system of Example 1, wherein the control circuit is configured to detect, at a first time, a first dropped voltage potential of the power source based on the motor transitioning to the on state, detect, at a second time subsequent to the first time, a second dropped voltage potential of the power source based on the motor transitioning to the on state, and predict a time that the voltage potential of the power source is expected to reach the power source lower threshold during a subsequent on state of the motor.

Example 5—The surgical system of Example 4, further comprising a display (3068) operably coupled to the control circuit, wherein the control circuit is configured to display the predicted time on the display.

Example 6—The surgical system of Examples 4 or 5, wherein the control circuit is configured to control the motor based on the prediction.

Example 7—The surgical system of Examples 1 or 2, wherein the control circuit is configured to set a power source upper threshold, detect a recovered voltage potential of the power source at the end of the second period, conduct a second comparison between the recovered voltage potential and the power source upper threshold, and control the motor based on the second comparison.

Example 8—The surgical system of Example 7, wherein the control circuit is configured to set a power source recovery threshold, wherein the control circuit is configured to maintain the motor in the off state for a recovery period based on the recovered voltage potential failing to reach the power source upper threshold, and wherein the recovery period corresponds to a time required for the voltage potential of the power source to recover to the power source recovery threshold.

Example 9—The surgical system of Example 7, wherein the control circuit is configured to transition the motor to the on state based on the dropped voltage potential being greater than the power source lower threshold.

Example 10—The surgical system of any one of Examples 1, 2, 4, or 5, wherein the control circuit is further configured set a power source upper threshold, detect, at a first time, a first recovered voltage potential of the power source based on the motor transitioning to the off state, detect, at a second time subsequent to the first time, a second recovered voltage potential of the power source based on the motor transitioning to the off state, and predict a time that the voltage potential of the power source is expected to reach the power source upper threshold during a subsequent off state on the motor.

Example 11—A surgical system (3030, 3050), comprising an end effector (3002, 3038), a motor (3056), a power source (3041) configured to power the motor, a timer (3069) configured to measure elapsed time, and a control circuit (3033, 3051) in operable communication with the motor and the timer. The end effector comprises a first jaw (3004, 3042) and a second jaw (3006, 3043) rotatable relative to the first jaw from an open position toward a closed position to capture tissue therebetween. The control circuit is configured to receive a first input, control the motor to drive a first motion at the end effector based on receiving the first input, monitor an elapsed period based on the first motion concluding, receive a second input, compare the elapsed period to a recovery time period based on receiving the second input, and perform an action based on the comparison.

Example 12—The surgical system of Example 11, wherein the action comprises abstaining from controlling the motor to drive a second motion at the end effector based on the elapsed period being less than the recovery time period.

Example 13—The surgical system of Example 12, further comprising a display (3068), wherein the action further comprises issuing a notification on the display based on the elapsed period being less than the recovery time period.

Example 14—The surgical system of any one of Examples 11-13, wherein the action comprises controlling the motor to drive a second motion at the end effector based on the elapsed period reaching or exceeding the recovery time period.

Example 15—The surgical system of Example 14, further comprising a display (3068), wherein the control circuit is further configured to issue a notification on the display based on the elapsed period reaching the recovery time period.

Example 16—The surgical system of Example 11, further comprising a voltage sensor (3039) configured to sense the voltage potential of the power source. The control circuit is further configured to set a recovery threshold, monitor a voltage potential of the power source based on the first motion concluding, compare the monitored voltage potential to the recovery threshold based on receiving the second input, and perform the action further based on the comparison of the monitored voltage potential to the recovery threshold based.

Example 17—The surgical system of Example 16, wherein the action comprises abstaining from controlling the motor to drive a second motion at the end effector based on the monitored voltage potential being less than the recovery threshold.

Example 18—The surgical system of Example 17, further comprising a display (3068), wherein the action further comprises issuing a notification on the display based on the monitored voltage potential being less than the recovery threshold.

Example 19—The surgical system of any one of Examples 16-18, wherein the action comprises controlling the motor to drive a second motion at the end effector based on the monitored voltage potential reaching or exceeding the recovery threshold.

Example 20—The surgical system of Example 19, further comprising a display (3068), wherein the control circuit is further configured to issue a notification on the display based on the monitored voltage potential reaching the recovery threshold.

Example 21—A surgical system comprising an end effector, a motor, a power source configured to power the motor, a voltage sensor configured to sense the voltage potential of the power source, and a control circuit in operable communication with the motor and the voltage sensor. The end effector comprises a first jaw and a second jaw rotatable relative to the first jaw from an open position toward a closed position to capture tissue therebetween. The motor is operable between an on state in which the motor drives a motion at the end effector and an off state in which the motor ceases to the motion at the end effector. The control circuit is configured to set a power source lower threshold, transition the motor to the on state for a first period, detect a dropped voltage potential of the power source at the end of the first period, conduct a first comparison between the dropped voltage potential and the power source lower threshold, and transition the motor to the off state for a second period based on the first comparison.

Example 22—The surgical system of Example 21, wherein the control circuit is configured to set a power source recovery threshold, wherein the second period comprises a recovery period based on the dropped voltage potential reaching or dropping below the power source lower threshold, and wherein the recovery period corresponds to a time required for the voltage potential of the power source to recover to the power source recovery threshold.

Example 23—The surgical system of Example 21, wherein the second period is identical to the first period based on the dropped voltage potential being greater than the power source lower threshold.

Example 24—The surgical system of Example 21, wherein the control circuit is configured to detect, at a first time, a first dropped voltage potential of the power source based on the motor transitioning to the on state, detect, at a second time subsequent to the first time, a second dropped voltage potential of the power source based on the motor transitioning to the on state, and predict a time that the voltage potential of the power source is expected to reach the power source lower threshold during a subsequent on state of the motor.

Example 25—The surgical system of Example 24, further comprising a display operably coupled to the control circuit, wherein the control circuit is configured to display the predicted time on the display.

Example 26—The surgical system of Example 24, wherein the control circuit is configured to control the motor based on the prediction.

Example 27—The surgical system of Example 21, wherein the control circuit is configured to set a power source upper threshold, detect a recovered voltage potential of the power source at the end of the second period, conduct a second comparison between the recovered voltage potential and the power source upper threshold, and control the motor based on the second comparison.

Example 28—The surgical system of Example 27, wherein the control circuit is configured to set a power source recovery threshold, wherein the control circuit is configured to maintain the motor in the off state for a recovery period based on the recovered voltage potential failing to reach the power source upper threshold, and wherein the recovery period corresponds to a time required for the voltage potential of the power source to recover to the power source recovery threshold.

Example 29—The surgical system of Example 27, wherein the control circuit is configured to transition the motor to the on state based on the dropped voltage potential being greater than the power source lower threshold.

Example 30—The surgical system of Example 21, wherein the control circuit is further configured set a power source upper threshold, detect, at a first time, a first recovered voltage potential of the power source based on the motor transitioning to the off state, detect, at a second time subsequent to the first time, a second recovered voltage potential of the power source based on the motor transitioning to the off state, and predict a time that the voltage potential of the power source is expected to reach the power source upper threshold during a subsequent off state on the motor.

Example 31—A surgical system comprising an end effector, a motor, a power source configured to power the motor, a voltage sensor configured to sense the voltage potential of the power source, and a control circuit in operable communication with the motor and the voltage sensor. The end effector comprises a first jaw and a second jaw rotatable relative to the first jaw from an open position toward a closed position to capture tissue therebetween. The control circuit is configured to set a recovery threshold, receive a first input, control the motor to drive a first motion at the end effector based on receiving the first input, monitor a voltage potential of the power source based on the first motion concluding, receive a second input, compare the monitored voltage potential to a recovery threshold based on receiving the second input, and perform an action based on the comparison.

Example 32—The surgical system of Example 31, wherein the action comprises abstaining from controlling the motor to drive a second motion at the end effector based on the monitored voltage potential being less than the recovery threshold.

Example 33—The surgical system of Example 32, further comprising a display, wherein the action further comprises issuing a notification on the display based on the monitored voltage potential being less than the recovery threshold.

Example 34—The surgical system of Example 31, wherein the action comprises controlling the motor to drive a second motion at the end effector based on the monitored voltage potential reaching or exceeding the recovery threshold.

Example 35—The surgical system of Example 31, further comprising a display, wherein the control circuit is further configured to issue a notification on the display based on the monitored voltage potential reaching the recovery threshold.

Example 36—A surgical system comprising an end effector, a motor, a power source configured to power the motor, a timer configured to measure elapsed time, and a control circuit in operable communication with the motor and the timer. The end effector comprises a first jaw and a second jaw rotatable relative to the first jaw from an open position toward a closed position to capture tissue therebetween. The control circuit is configured to receive a first input, control the motor to drive a first motion at the end effector based on receiving the first input, monitor an elapsed period based on the first motion concluding, receive a second input, compare the elapsed period to a recovery time period based on receiving the second input, and perform an action based on the comparison.

Example 37—The surgical system of Example 36, wherein the action comprises abstaining from controlling the motor to drive a second motion at the end effector based on the elapsed period being less than the recovery time period.

Example 38—The surgical system of Example 37, further comprising a display, wherein the action further comprises issuing a notification on the display based on the elapsed period being less than the recovery time period.

Example 39—The surgical system of Example 36, wherein the action comprises controlling the motor to drive a second motion at the end effector based on the elapsed period reaching or exceeding the recovery time period.

Example 40—The surgical system of Example 36, further comprising a display, wherein the control circuit is further configured to issue a notification on the display based on the elapsed period reaching the recovery time period.

While several configurations have been described, additional modifications are within the scope of the present disclosure, which is intended to cover any variations, uses, or adaptations of the disclosed configurations using its general principles.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. The instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” or “control system” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in one aspect. Thus, appearances of the phrases “in one aspect,” “in an aspect,” “in an exemplification,” and “in one exemplification” in various places throughout the specification are not necessarily all referring to the same aspect. The particular features, structures or characteristics may be combined in any suitable manner in various aspects.

It is worthy to note that any reference numbers included in the appended claims are used to reference exemplary embodiments/elements described in the present disclosure. Accordingly, any such reference numbers are not meant to limit the scope of the subject matter recited in the appended claims.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1-20. (canceled)

21. A surgical system, comprising:
an end effector, comprising:
a first jaw; and
a second jaw rotatable relative to the first jaw from an open position toward a closed position to capture tissue therebetween;
a motor, operable between:
an on state in which the motor drives a motion at the end effector; and
an off state in which the motor ceases to the motion at the end effector;
a power source configured to power the motor;
a voltage sensor configured to sense the voltage potential of the power source; and
a control circuit in operable communication with the motor and the voltage sensor, wherein the control circuit is configured to:
set a power source lower threshold;
transition the motor to the on state for a first period;
detect a dropped voltage potential of the power source at the end of the first period;
conduct a first comparison between the dropped voltage potential and the power source lower threshold; and
transition the motor to the off state for a second period based on the first comparison.

22. The surgical system of claim 21, wherein the control circuit is configured to set a power source recovery threshold, wherein the second period comprises a recovery period based on the dropped voltage potential reaching or dropping below the power source lower threshold, and wherein the recovery period corresponds to a time required for the voltage potential of the power source to recover to the power source recovery threshold.

23. The surgical system of claim 21, wherein the second period is identical to the first period based on the dropped voltage potential being greater than the power source lower threshold.

24. The surgical system of claim 21, wherein the control circuit is configured to:
detect, at a first time, a first dropped voltage potential of the power source based on the motor transitioning to the on state;
detect, at a second time subsequent to the first time, a second dropped voltage potential of the power source based on the motor transitioning to the on state; and
predict a time that the voltage potential of the power source is expected to reach the power source lower threshold during a subsequent on state of the motor.

25. The surgical system of claim 24, further comprising a display operably coupled to the control circuit, wherein the control circuit is configured to display the predicted time on the display.

26. The surgical system of claim 24, wherein the control circuit is configured to control the motor based on the prediction.

27. The surgical system of claim 21, wherein the control circuit is configured to:
set a power source upper threshold;
detect a recovered voltage potential of the power source at the end of the second period;
conduct a second comparison between the recovered voltage potential and the power source upper threshold; and
control the motor based on the second comparison.

28. The surgical system of claim 27, wherein the control circuit is configured to set a power source recovery threshold, wherein the control circuit is configured to maintain the motor in the off state for a recovery period based on the recovered voltage potential failing to reach the power source upper threshold, and wherein the recovery period corresponds to a time required for the voltage potential of the power source to recover to the power source recovery threshold.

29. The surgical system of claim 27, wherein the control circuit is configured to transition the motor to the on state based on the dropped voltage potential being greater than the power source lower threshold.

30. The surgical system of claim 21, wherein the control circuit is further configured:
set a power source upper threshold;
detect, at a first time, a first recovered voltage potential of the power source based on the motor transitioning to the off state;
detect, at a second time subsequent to the first time, a second recovered voltage potential of the power source based on the motor transitioning to the off state; and
predict a time that the voltage potential of the power source is expected to reach the power source upper threshold during a subsequent off state on the motor.

31. A surgical system, comprising:
an end effector, comprising:
a first jaw; and
a second jaw rotatable relative to the first jaw from an open position toward a closed position to capture tissue therebetween;
a motor;
a power source configured to power the motor;
a voltage sensor configured to sense the voltage potential of the power source; and
a control circuit in operable communication with the motor and the voltage sensor, wherein the control circuit is configured to:
set a recovery threshold;
receive a first input;
control the motor to drive a first motion at the end effector based on receiving the first input;
monitor a voltage potential of the power source based on the first motion concluding;
receive a second input;
compare the monitored voltage potential to a recovery threshold based on receiving the second input; and
perform an action based on the comparison.

32. The surgical system of claim 31, wherein the action comprises abstaining from controlling the motor to drive a second motion at the end effector based on the monitored voltage potential being less than the recovery threshold.

33. The surgical system of claim 32, further comprising a display, wherein the action further comprises issuing a notification on the display based on the monitored voltage potential being less than the recovery threshold.

34. The surgical system of claim 31, wherein the action comprises controlling the motor to drive a second motion at the end effector based on the monitored voltage potential reaching or exceeding the recovery threshold.

35. The surgical system of claim 31, further comprising a display, wherein the control circuit is further configured to issue a notification on the display based on the monitored voltage potential reaching the recovery threshold.

36. A surgical system, comprising:
an end effector, comprising:
a first jaw; and
a second jaw rotatable relative to the first jaw from an open position toward a closed position to capture tissue therebetween;
a motor;
a power source configured to power the motor;
a timer configured to measure elapsed time; and
a control circuit in operable communication with the motor and the timer, wherein the control circuit is configured to:
receive a first input;
control the motor to drive a first motion at the end effector based on receiving the first input;
monitor an elapsed period based on the first motion concluding;
receive a second input;
compare the elapsed period to a recovery time period based on receiving the second input; and
perform an action based on the comparison.

37. The surgical system of claim 36, wherein the action comprises abstaining from controlling the motor to drive a second motion at the end effector based on the elapsed period being less than the recovery time period.

38. The surgical system of claim 37, further comprising a display, wherein the action further comprises issuing a notification on the display based on the elapsed period being less than the recovery time period.

39. The surgical system of claim 36, wherein the action comprises controlling the motor to drive a second motion at the end effector based on the elapsed period reaching or exceeding the recovery time period.

40. The surgical system of claim 36, further comprising a display, wherein the control circuit is further configured to issue a notification on the display based on the elapsed period reaching the recovery time period.
